(19) United States
(12) Patent Application Publication
(10) Pub. No.: US 2023/0414787 A1
(43) Pub. Date: Dec. 28, 2023

(54) GENE KNOCK-OUT FOR TREATMENT OF GLAUCOMA

(71) Applicant: University of Iowa Research Foundation, Iowa City, IA (US)

(72) Inventors: Qihong Zhang, Iowa City, IA (US); Charles C. Searby, Iowa City, IA (US); Val C. Sheffield, Iowa City, IA (US); Calvin S. Carter, Iowa City, IA (US); Sunny C. Huang, Iowa City, IA (US)

(21) Appl. No.: 18/023,259

(22) PCT Filed: Aug. 27, 2021
(86) PCT No.: PCT/US2021/048018

§ 371 Date: Feb. 24, 2023

Related U.S. Application Data
(60) Provisional application No. 63/071,161, filed on Aug. 27, 2020.

(51) Int. Cl. A61K 48/00; C12N 15/86; C12N 9/22; C07K 14/47; A61P 27/06

(52) U.S. Cl. CPC A61K 48/0075; C12N 15/86; C12N 9/22; C07K 14/47; A61P 27/06

(57) ABSTRACT

A vector and methods of using the vector for, for example, to prevent, inhibit or treat glaucoma, decrease intraocular pressure or to reduce MYOC expression in a mammal, are provided.

### CROSS-REFERENCE TO RELATED APPLICATIONS

This application is a U.S. National Stage Filing under 35 U.S.C. 371 from International Application No. PCT/US2021/048018, filed on Aug. 27, 2021, and published as WO 2022/047201 on Mar. 3, 2022, which application claims the benefit of the filing date of U.S. application No. 63/071,161, filed on Aug. 27, 2020, the disclosures of which are incorporated by reference herein.

### INCORPORATION BY REFERENCE OF SEQUENCE LISTING

A Sequence Listing is provided herewith as a text file, “2365982.TXT” created on Sep. 7, 2023 and having a size of 10,471 bytes. The contents of the text file are incorporated by reference herein in their entirety.

### BACKGROUND

Glaucoma is a leading cause of irreversible vision loss and blindness in the world, affecting approximately 80 million individuals. Age is a major risk factor for the development of glaucoma, so the prevalence of glaucoma will continue to increase with an aging population. Elevated IOP is the most important causative risk factor and is due to damage to the trabecular meshwork (TM) and aqueous humor outflow pathway. A variety of glaucomatous insults damage the TM including genetic variations (MYOC mutations), glucocorticoids (GCs), and the profibrotic cytokine TGFβ2. Studies of these factors have led to discovery of common pathogenic mechanisms (Zode et al., 2011; Zode et al., 2014; Kasetti et al., 2018).

Current glaucoma therapies target lowering IOP, but these pharmacological and surgical approaches do not directly intervene in glaucoma TM pathogenesis, do not cure glaucoma and lose efficacy over time. In addition, topical ocular pharmaceutical therapies require glaucoma patients to administer eye drops one to multiple times per day. Unfortunately, patient compliance to this therapeutic approach is often poor, further complicating long term care of glaucoma patients (Joseph & Pasquale, 2017). Molecular editing of genes involved in pathogenic damage to cells and tissues holds promise in molecular medicine (Ran et al., 2013; Razzouk, 2018). Examples of attempts to cure diverse diseases with genome editing include inherited cardiovascular diseases (Kaur et al., 2018), viral infections (Chen et al., 2018), cancers, and eye diseases (Mollanoori & Teimourian, 2018).

### SUMMARY

The disclosure provides composition and methods for genome editing, e.g., using the CRISPR-Cas9 system and guide-RNA (gRNA) specific for the myocilin gene, to, in one example, inactivate the myocilin gene, thereby providing a one-time “cure”. That editing normalizes the TM and IOP of glaucoma patients. In particular, transduction of the TM with a vector encoding CRISPR-Cas9 and gRNA knocks out expression of MYOC in the TM which in turn reverses mutant MYOC induced ocular hypertension. As described herein, two CRISPR guide-RNAs that specifically target the human myocilin gene were identified that have high efficiency in inactivating the human myocilin gene in human trabecular meshwork cells (e.g., NTM5 and GTM3 cells). Sequences for the guide RNAs for human myocilin include but are not limited to; myoc-cut1: GTCAGTCATCCATAACTTACA (SEQ ID NO:1), myoc-cut2: GACCAGCTGGAAACCCAAACCA (SEQ ID NO:2), myoc-cut3 GGCTCCAGAGAAGTTTCTACG (SEQ ID NO:3), and myoc-cut4 GCCAAAGTGTCCAAATTCCACG (SEQ ID NO:4), or a sequence having at least 90%, 92%, 95%, 96%, 98% or more nucleotide sequence identity thereto. e.g., a sequence with 1, 2, 3, 4 or 5 nucleotide substitutions. In one embodiment, the guide RNA employed in the vector or methods is not GGCCTGCCTGGTGTGGGATG (SEQ ID NO:5). In one embodiment, the vector encodes saCas9. In one embodiment, the vector does not encode spCas9. In one embodiment, the vector is an adeno-associated virus (AAV) vector. In one embodiment, the vector is an adeno-associated virus (AAV) serotype 2/2. In one embodiment, the vector is an adeno-associated virus (AAV) serotype 2/5. In one embodiment, the vector is not an adenovirus vector.

In one embodiment, the administration of the vector allows for CRISPR based editing, e.g., genetic modifications that result in decreased activity, for example, decreased transcription or inactivation, of a myocilin gene mutation in patients with MYOC glaucoma. In one embodiment, a patient with MYOC glaucoma has a mutation in the C-terminal coding region of MYOC. In one embodiment, the mutation results in a mis-folded MYOC, e.g., as a result of an amino acid substitution. In one embodiment, the editing results in decreased expression of MYOC, e.g., a mutant MYOC. In one embodiment, the editing results in a truncated MYOC, e.g., as a result of a nucleotide substitution that yields a stop codon or a splice site. In one embodiment, the administration of the vector modifies a wild-type MYOC gene, thereby decreasing expression of a wild-type MYOC. In one embodiment, the glaucoma is open angle glaucoma. In one embodiment, both copies of the MYOC gene in a TM cell are modified. In one embodiment, only one copy of the MYOC gene in a TM cell is modified. In one embodiment, the vector has a guide-RNA sequence that is targeted to the N-terminal coding region of MYOC, e.g., to the N-terminal 250, 200, 150, 100, 50 or 25 amino acids of MYOC.

In one embodiment, a method to decrease MYOC expression in TM cells is provided. In one embodiment, a method to decrease IOP in a mammalian eye is provided. The methods employ CRISPR based mutation, e.g., of a myocilin gene, so that gene expression is reduced or eliminated. In one embodiment, gene expression is reduced or eliminated by introducing an in-frame stop codon, e.g., via nucleotide substitution(s), frame shift, insertion or a deletion. In one embodiment the CRISPR based mutation is an indel that results in a frameshift. In one embodiment, the method employs Cas, for example, comprises Streptococcus pyogenes (SpCas9). Staphylococcus aureus (SaCas9), Streptococcus thermophilus (StCas9), Neisseria meningitidis (NmCas9), Francisella novicida (FnCas9), Campylobacter jejuni (CjCas9), CasX and CasY, Cas12a (Cpf1), Cas14a, eSpCas9, SpCas9-HF1, HypaCas9, FokI-Fused dCas9, or xCas9.

### BRIEF DESCRIPTION OF THE FIGURES

FIG. 1. Transduction of primary human TM cells using various AAVs. Human primary TM cells (n=2 strains) were transduced with several AAV serotypes. GFP expression was examined. Scale bar=200 μm.

FIG. 2. AAV-GFP transduction in mouse eyes. C57 mice (n=3) were transduced with AAV2-GFP or AAV5-GFP via intracameral or intravitreal injections. GFP expression was examined in the anterior segment tissues after 2 weeks of injection. Scale bar=50 μm.

FIGS. 3A-3C. CRISPR-Cas9 lowers IOP and prevents further glaucomatous damage. (A) Ad5-crMYOC (2×10⁷ pfu/eye) lowers IOP in older Tg-MYOC^{Y437H} mice (n=9). (B) Preserved outflow facility in eyes of Tg-MYOCY437H mice after Ad5-crMYOC treatment (n=7). (C) Improved RGC function measured by pERG in eyes of 4- to 5-mo-old Tg-MYOC^{Y437H} mice after Ad5-crMYOC treatment n=13). *P<0.05; ***P<0.001; ****P<0.0001.

FIGS. 4A-4B. CRISPR efficiency in vivo in mice and ex vivo in human eyes. A) Ad5-crMYOC treatment reduces myocilin, KDEL and CHOP levels in the TM (box) of Tg-MYOC^{Y437H} mice (n=3). C, cornea CB, ciliary body. (Scale bar: 50 μm). B) Ad5-crMYOC-mediated myocilin knockdown in a human donor eyes. Coomassie blue shows relatively equal loading, n=2.

FIGS. 5A-5C. DEX-induced human ocular hypertension. Pairs of human donor eyes were set up for anterior segment POC as previously described (Clark et al., 1995). One eye of each pair was treated with DEX (100 nM), while the other eye received vehicle (0.1% ethanol). The eyes received constant flow perfusion. IOPs were monitored over 14 days. (A) Pair of non-responder eyes without elevated IOP. (B) Pair of responder eyes that develop ocular hypertension. (C) DEX treatment caused a significant increase in IOP (>5 mm Hg) in 40% of the perfusion cultured eyes. Closed circles=DEX responder eyes; open circles=non-responder eyes.

FIGS. 6A-6E. Development of novel mouse model of GC-induced ocular hypertension. A) Nighttime IOP measurements of DEX (n=20) versus Veh (n=12) treated mice show significant IOP elevation. B) Reduced conventional outflow facility (C) in DEX (n=13) treated mice compared to Veh (n=12) treated mice. C) DEX increased fibronectin and a-smooth muscle actin (SMA) in TM tissues. TM is shown by arrow. D) Pattern ERG amplitudes as a function of RGCs in 10-weeks Veh or DEX injected mice is shown. N=6. E) RGC loss in 12-weeks DEX-treated mice.

FIG. 7. Exemplary AAV vector.

### DETAILED DESCRIPTION

### Definitions

As used herein, “individual” (as in the subject of the treatment) means a mammal. Mammals include, for example, humans; non-human primates, e.g., apes and monkeys; and non-primates, e.g., dogs, cats, rats, mice, cattle, pigs, horses, sheep, and goats. Non-mammals include, for example, fish and birds.

The term “disease” or “disorder” are used interchangeably, and are used to refer to diseases or conditions wherein lack of or reduced amounts of a specific gene product, e.g., a lysosomal storage enzyme, plays a role in the disease such that a therapeutically beneficial effect can be achieved by supplementing, e.g., to at least 1% of normal levels.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of symptoms associated with a disorder or disease, “inhibiting” means inhibition of further progression or worsening of the symptoms associated with the disorder or disease, and “preventing” refers to prevention of the symptoms associated with the disorder or disease.

As used herein, an “effective amount” or a “therapeutically effective amount” of an agent, e.g., a recombinant AAV encoding a gene product, refers to an amount of the agent that alleviates, in whole or in part, symptoms associated with the disorder or condition, or halts or slows further progression or worsening of those symptoms, or prevents or provides prophylaxis for the disorder or condition, e.g., an amount that is effective to prevent, inhibit or treat in the individual one or more symptoms.

In particular, a “therapeutically effective amount” refers to an amount effective, at dosages and for periods of time necessary, to achieve the desired therapeutic result. A therapeutically effective amount is also one in which any toxic or detrimental effects of the agent(s) are outweighed by the therapeutically beneficial effects.

A “vector” as used herein refers to a macromolecule or association of macromolecules that comprises or associates with a polynucleotide and which can be used to mediate delivery of the polynucleotide to a cell, either in vitro or in vivo. Illustrative vectors include, for example, plasmids, viral vectors, liposomes and other gene delivery vehicles. The polynucleotide to be delivered, sometimes referred to as a “target polynucleotide” or “transgene,” may comprise a coding sequence of interest in gene therapy (such as a gene encoding a protein of therapeutic interest) and/or a selectable or detectable marker.

“AAV” is adeno-associated virus, and may be used to refer to the virus itself or derivatives thereof. The term covers all subtypes, serotypes and pseudotypes, and both naturally occurring and recombinant forms, except where required otherwise. As used herein, the term “serotype” refers to an AAV which is identified by and distinguished from other AAVs based on its binding properties. e.g., there are eleven serotypes of AAVs, AAV1-AAV11, including AAV2, AAV5, AAV6, AAV8, AAV9 and AAVrh10, and the term encompasses pseudotypes with the same binding properties. Thus, for example, AAV9 serotypes include AAV with the binding properties of AAV9, e.g., a pseudotyped AAV comprising AAV9 capsid and a rAAV genome which is not derived or obtained from AAV9 or which genome is chimeric. The abbreviation “rAAV” refers to recombinant adeno-associated virus, also referred to as a recombinant AAV vector (or “rAAV vector”).

An “AAV virus” refers to a viral particle composed of at least one AAV capsid protein and an encapsidated polynucleotide. If the particle comprises a heterologous polynucleotide (i.e., a polynucleotide other than a wild-type AAV genome such as a transgene to be delivered to a mammalian cell), it is typically referred to as “rAAV”. An AAV “capsid protein” includes a capsid protein of a wild-type AAV, as well as modified forms of an AAV capsid protein which are structurally and or functionally capable of packaging a rAAV genome and bind to at least one specific cellular receptor which may be different than a receptor employed by wild type AAV. A modified AAV capsid protein includes a chimeric AAV capsid protein such as one having amino acid sequences from two or more serotypes of AAV, e.g., a capsid protein formed from a portion of the capsid protein from AAV9 fused or linked to a portion of the capsid protein from AAV-2, and a AAV capsid protein having a tag or other detectable non-AAV capsid peptide or protein fused or linked to the AAV capsid protein, e.g., a portion of an antibody molecule which binds a receptor other than the receptor for AAV9, such as the transferrin receptor, may be recombinantly fused to the AAV9 capsid protein.

A “pseudotyped” rAAV is an infectious virus having any combination of an AAV capsid protein and an AAV genome. Capsid proteins from any AAV serotype may be employed with a rAAV genome which is derived or obtainable from a wild-type AAV genome of a different serotype or which is a chimeric genome, i.e., formed from AAV DNA from two or more different serotypes, e.g., a chimeric genome having 2 inverted terminal repeats (ITRs), each ITR from a different serotype or chimeric ITRs. The use of chimeric genomes such as those comprising ITRs from two AAV serotypes or chimeric ITRs can result in directional recombination which may further enhance the production of transcriptionally active intermolecular concatamers. Thus, the 5′ and 3′ ITRs within a rAAV vector of the invention may be homologous, i.e., from the same serotype, heterologous, i.e., from different serotypes, or chimeric, i.e., an ITR which has ITR sequences from more than one AAV serotype.

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. “Affinity” refers to the strength of binding, increased binding affinity being correlated with a lower Kd.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), preferably between about 100 and 1,000 nucleotides in length (or any integer therebetween), more preferably between about 200 and 500 nucleotides in length.

A “homologous, non-identical sequence” refers to a first sequence which shares a degree of sequence identity with a second sequence, but whose sequence is not identical to that of the second sequence. For example, a polynucleotide comprising the wild-type sequence of a mutant gene is homologous and non-identical to the sequence of the mutant gene. In certain embodiments, the degree of homology between the two sequences is sufficient to allow homologous recombination therebetween, utilizing normal cellular mechanisms. Two homologous non-identical sequences can be any length and their degree of non-homology can be as small as a single nucleotide (e.g., for correction of a genomic point mutation by targeted homologous recombination) or as large as 10 or more kilobases (e.g., for insertion of a gene at a predetermined ectopic site in a chromosome). Two polynucleotides comprising the homologous non-identical sequences need not be the same length. For example, an exogenous polynucleotide (i.e., donor polynucleotide) of between 20 and 10,000 nucleotides or nucleotide pairs can be used.

A “target site” or “target sequence” is a nucleic acid sequence that defines a portion of a nucleic acid to which a binding molecule will bind, provided sufficient conditions for binding exist.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (e.g., liposomes, including neutral and cationic lipids), electroporation, direct injection, cell fusion, particle bombardment, calcium phosphate co-precipitation. DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid.

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence.

### The CRISPR/Cas System

The Type II CRISPR is a well characterized system that carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation,’ (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system. The primary products of the CRISPR loci appear to be short RNAs that contain the invader targeting sequences, and are termed guide RNAs

“Cas1” polypeptide refers to CRISPR associated (Cas) protein1. Cas1 (COG1518 in the Clusters of Orthologous Group of proteins classification system) is the best marker of the CRISPR-associated systems (CASS). Based on phylogenetic comparisons, seven distinct versions of the CRISPR-associated immune system have been identified (CASS1-7). Cas1 polypeptide used in the methods described herein can be any Cas1 polypeptide present in any prokaryote. In certain embodiments, a Cas1 polypeptide is a Cas1 polypeptide of an archaeal microorganism. In certain embodiments, a Cas1 polypeptide is a Cas1 polypeptide of a Euryarchaeota microorganism. In certain embodiments, a Cas1 polypeptide is a Cas1 polypeptide of a Crenarchaeota microorganism. In certain embodiments, a Cas1 polypeptide is a Cas1 polypeptide of a bacterium. In certain embodiments, a Cas1 polypeptide is a Cas1 polypeptide of a gram negative or gram positive bacteria. In certain embodiments, a Cas1 polypeptide is a Cas1 polypeptide of Pseudomonas aeruginosa. In certain embodiments, a Cas1 polypeptide is a Cas1 polypeptide of Aquifex aeolicus. In certain embodiments, a Cas1 polypeptide is a Cas1 polypeptide that is a member of one of CASs1-7. In certain embodiments, Cas1 polypeptide is a Cas t polypeptide that is a member of CASS3. In certain embodiments, a Cas1 polypeptide is a Cas1 polypeptide that is a member of CASS7. In certain embodiments, a Cas1 polypeptide is a Cast polypeptide that is a member of CASS3 or CASS7.

In some embodiments, a Cas1 polypeptide is encoded by a nucleotide sequence provided in GenBank at, e.g., GeneID number: 2781520, 1006874, 9001811, 947228, 3169280, 2650014, 1175302, 3993120, 4380485, 906625, 3165126, 905808, 1454460, 1445886, 1485099, 4274010, 888506, 3169526, 997745, 897836, or 1193018 and/or an amino acid sequence exhibiting homology (e.g., greater than 80%, 90 to 99% including 91%, 92%, 93%, 94%, 95%, 96%, 97%, 98% or 99%) to the amino acids encoded by these polynucleotides and which polypeptides function as Cast polypeptides.

There are three types of CRISPR/Cas systems which all incorporate RNAs and Cas proteins. Types I and III both have Cas endonucleases that process the pre-crRNAs, that, when fully processed into crRNAs, assemble a multi-Cas protein complex that is capable of cleaving nucleic acids that are complementary to the crRNA.

In type II CRISPR/Cas systems, crRNAs are produced using a different mechanism where a trans-activating RNA (tracrRNA) complementary to repeat sequences in the pre-crRNA, triggers processing by a double strand-specific RNase III in the presence of the Cas9 protein. Cas9 is then able to cleave a target DNA that is complementary to the mature crRNA however cleavage by Cas 9 is dependent both upon base-pairing between the crRNA and the target DNA, and on the presence of a short motif in the crRNA referred to as the PAM sequence (protospacer adjacent motif)). In addition, the tracrRNA must also be present as it base pairs with the crRNA at its 3′ end, and this association triggers Cas9 activity.

The Cas9 protein has at least two nuclease domains: one nuclease domain is similar to a HNH endonuclease, while the other resembles a Ruv endonuclease domain. The HNH-type domain appears to be responsible for cleaving the DNA strand that is complementary to the crRNA while the Ruv domain cleaves the non-complementary strand.

The requirement of the crRNA-tracrRNA complex can be avoided by use of an engineered “single-guide RNA” (sgRNA) that comprises the hairpin normally formed by the annealing of the crRNA and the tracrRNA (see Jinek, et al. (2012) Science 337:816 and Cong et al. (2013) Sciencexpress/10.1126/science.1231143). In S. pyrogenes, the engineered tracrRNA:crRNA fusion, or the sgRNA, guides Cas9 to cleave the target DNA when a double strand RNA:DNA heterodimer forms between the Cas associated RNAs and the target DNA. This system comprising the Cas9 protein and an engineered sgRNA

“Cas polypeptide” encompasses a full-length Cas polypeptide, an enzymatically active fragment of a Cas polypeptide, and enzymatically active derivatives of a Cas polypeptide or fragment thereof. Suitable derivatives of a Cas polypeptide or a fragment thereof include but are not limited to mutants, fusions, covalent modifications of Cas protein or a fragment thereof.

### RNA Components of CRISPR/Cas

The Cas9 related CRISPR/Cas system comprises two RNA non-coding components: tracrRNA and a pre-crRNA array containing nuclease guide sequences (spacers) interspaced by identical direct repeats (DRs). To use a CRISPR/Cas system to accomplish genome engineering, both functions of these RNAs must be present (see Cong, et al. (2013) Sciencexpress 1/10.1126/science 1231143). In some embodiments, the tracrRNA and pre-crRNAs are supplied via separate expression constructs or as separate RNAs. In other embodiments, a chimeric RNA is constructed where an engineered mature crRNA (conferring target specificity) is fused to a tracrRNA (supplying interaction with the Cas9) to create a chimeric cr-RNA-tracrRNA hybrid (also termed a single guide RNA). (see Jinek, ibid and Cong, ibid).

Chimeric or sgRNAs can be engineered to comprise a sequence complementary to any desired target. The RNAs comprise 22 bases of complementarity to a target and of the form G[n19], followed by a protospacer-adjacent motif (PAM) of the form NGG. Thus, in one method, sgRNAs can be designed by utilization of a known ZFN target in a gene of interest by (i) aligning the recognition sequence of the ZFN heterodimer with the reference sequence of the relevant genome (human, mouse, or of a particular plant species); (ii) identifying the spacer region between the ZFN half-sites; (iii) identifying the location of the motif G[N20]GG that is closest to the spacer region (when more than one such motif overlaps the spacer, the motif that is centered relative to the spacer is chosen); (iv) using that motif as the core of the sgRNA. This method advantageously relies on proven nuclease targets. Alternatively, sgRNAs can be designed to target any region of interest simply by identifying a suitable target sequence that conforms to the G[n20]GG formula.

### Glaucoma

Glaucoma is a leading cause of irreversible vision loss and blindness in the world, affecting approximately 80 million individuals (Tham et al., 2014). Over ten percent of individuals with glaucoma become bilaterally blind due to this disease (Peters et al., 2013). The molecular mechanisms responsible for glaucomatous damage to the TM have been studied and relevant ex vivo human and in vivo mouse models of ocular hypertension (OHT) and glaucoma have been prepared (Clark et al., 1995; Zode et al., 2014; Patel et al., 2017; Shepard et al., 2010; Clark et al., 2005; Fleenor et al., 2006; McDowell et al., 2013; Fuchshofer et al., 2003). Regardless of the initiating insult (e.g., mutant MYOC, glucocorticoids, TGFβ2), it appears that endoplasmic reticulum (ER) stress is involved in TM damage and IOP elevation (Zode et al., 2011; Zode et al., 2014; Kasetti et al., 2018). In fact, specific markers for ER stress are elevated in TM tissues of primary open angle glaucoma (POAG) human donor eyes (Peters et al., 2015). As described herein below, genome editing was used with the CRISPR-Cas9 system to specifically target glaucomatous molecular pathways in the TM to provide a one-time “cure”. This is a disease modifying therapy that normalizes the TM and eliminates the current issues with patient noncompliance.

Glaucomatous damage to the TM is responsible for increased aqueous humor outflow resistance and elevated IOP. Elevated IOP is a major causative risk factor for the development and progression of glaucoma. Major molecular pathogenic pathways have been discovered that are responsible for glaucomatous damage to the TM and elevated IOP, including mutant MYOC and the unfolded protein response (UPR). Specifically, chronic ER stress induces transcriptional factors including ATF4 and CHOP in human post-mortem glaucomatous TM tissues and also in TM tissues of mouse models of glaucoma (Zode et al., 2011; Zode et al., 2014; Kasetti et al., 2018; Peters et al., 2015). In vivo mouse glaucoma models (TgMYOCY437H, DEX-OHT, TGFβ2-OHT), as well as ex vivo human POC models of OHT (DEX-OHT, TGFβ2-OHT) (Clark et al., 1995; Clark et al., 2005; Fleenor et al., 2006) have been developed.

Targeting IOP to inhibit the development and progression of glaucoma damage has been well documented in multiple clinical trials (Kass et al., 2002; Heiji et al., 2002; AGIS, 2000). It is also well recognized that glaucomatous damage to the TM is responsible for this elevated IOP. Mutations in MYOC are responsible for a subset of POAG (Stone et al., 1997; Alward et al., 1998), and gain-of-function MYOC mutations result in non-secretion of MYOC (Jacobson et al., 2001), and non-secretion of mutant MYOC leads to ER stress in the TM of a transgenic mouse model of MYOC glaucoma (Zode et al., 2011). There has been seven decades of reports that prolonged GC therapy can cause GC-induced OHT and iatrogenic glaucoma (Clark, 1995; Razeghinejad & Katz, 2012). In vivo mouse models as well as ex vivo in human POC anterior segment models of GC-induced OHT have independently validated this important and significant side effect of GC therapy (Clark et al., 1995; Zode et al., 2014; Patel et al., 2017; Clark et al., 2005). Numerous studies have demonstrated increased expression of the profibrotic cytokine, TGFβ2, in the aqueous humor and TM of POAG eyes (Inatani et al., 2001; Picht et al., 2001; Ochiai & Ochiai 2002; Tovar-Vidales et al., 2011), and this elevated TGFβ2 expression alters the TM cytoskeleton (Montecchi-Palmer et al., 2017) and extracellular matrix (ECM) (Lutjen-Drecoll, 2005). There is evidence for increased UPR and ER stress within the TM in MYOC, GC. TGFβ2 models as well as in the TM of POAG eyes (Stone et al., 1997; Zode et al., 2011; Zode et al., 2011; Zode et al., 2014; Kasetti et al., 2018; Peters et al., 2015). Therefore, targeting UPR and ER stress in the TM may reduce IOP and prevent glaucomatous optic neuropathy and retinopathy in all these forms of glaucoma.

### Gene Delivery Vectors

Gene delivery vectors within the scope of the invention include, but are not limited to, isolated nucleic acid, e.g., plasmid-based vectors which may be extrachromosomally maintained, and viral vectors, e.g., recombinant adenovirus, retrovirus, lentivirus, herpesvirus, poxvirus, papilloma virus, or adeno-associated virus, including viral and non-viral vectors which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes. Exemplary viral gene delivery vectors are described below. Gene delivery vectors may be administered via any route including, but not limited to, intracranial, intrathecal, intramuscular, buccal, rectal, intravenous or intracoronary administration, and transfer to cells may be enhanced using electroporation and/or iontophoresis, and/or scaffolding such as extracellular matrix or hydrogels, e.g., a hydrogel patch. In one embodiment, a permeation enhancer is not employed to enhance indirect delivery to the CNS.

### Retroviral Vectors

Retroviral vectors exhibit several distinctive features including their ability to stably and precisely integrate into the host genome providing long-term transgene expression. These vectors can be manipulated ex vivo to eliminate infectious gene particles to minimize the risk of systemic infection and patient-to-patient transmission. Pseudotyped retroviral vectors can alter host cell tropism.

### Lentiviruses

Lentiviruses are derived from a family of retroviruses that include human immunodeficiency virus and feline immunodeficiency virus. However, unlike retroviruses that only infect dividing cells, lentiviruses can infect both dividing and nondividing cells. For instance, lentiviral vectors based on human immunodeficiency virus genome are capable of efficient transduction of cardiac myocytes in vivo. Although lentiviruses have specific tropisms, pseudotyping the viral envelope with vesicular stomatitis virus yields virus with a broader range (Schnepp et al., Meth. Mol. Med., 69:427 (2002)).

### Adenoviral Vectors

Adenoviral vectors may be rendered replication-incompetent by deleting the early (E1A and E1B) genes responsible for viral gene expression from the genome and are stably maintained into the host cells in an extrachromosomal form. These vectors have the ability to transfect both replicating and nonreplicating cells and, in particular, these vectors have been shown to efficiently infect cardiac myocytes in vivo, e.g., after direction injection or perfusion. Adenoviral vectors have been shown to result in transient expression of therapeutic genes in vivo, peaking at 7 days and lasting approximately 4 weeks. The duration of transgene expression may be improved in systems utilizing neural specific promoters. In addition, adenoviral vectors can be produced at very high titers, allowing efficient gene transfer with small volumes of virus.

### Adeno-Associated Virus Vectors

Recombinant adeno-associated viruses (rAAV) are derived from nonpathogenic parvoviruses, evoke essentially no cellular immune response, and produce transgene expression lasting months in most systems. Moreover, like adenovirus, adeno-associated virus vectors also have the capability to infect replicating and nonreplicating cells and are believed to be nonpathogenic to humans. Moreover, they appear promising for sustained cardiac gene transfer (Hoshijima et al., Nat. Med., 8:864 (2002); Lynch et al., Circ. Res., 80:197 (1997)).

AAV vectors include but are not limited to AAV1, AAV2, AAV5, AAV7, AAV8, AAV9 or AAVrh.10.

### Plasmid DNA Vectors

Plasmid DNA is often referred to as “naked DNA” to indicate the absence of a more elaborate packaging system. Direct injection of plasmid DNA to myocardial cells in vivo has been accomplished. Plasmid-based vectors are relatively nonimmunogenic and nonpathogenic, with the potential to stably integrate in the cellular genome, resulting in long-term gene expression in postmitotic cells in vivo. For example, expression of secreted angiogenesis factors after muscle injection of plasmid DNA, despite relatively low levels of focal transgene expression, has demonstrated significant biologic effects in animal models and appears promising clinically (Isner, Nature, 415:234 (2002)). Furthermore, plasmid DNA is rapidly degraded in the blood stream; therefore, the chance of transgene expression in distant organ systems is negligible. Plasmid DNA may be delivered to cells as part of a macromolecular complex, e.g., a liposome or DNA-protein complex, and delivery may be enhanced using techniques including electroporation.

Exemplary rAAV Vectors

Adeno-associated viruses of any serotype are suitable to prepare rAAV, since the various serotypes are functionally and structurally related, even at the genetic level. All AAV serotypes apparently exhibit similar replication properties mediated by homologous rep genes; and all generally bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to ITRs. The similar infectivity patterns also suggest that the replication functions in each serotype are under similar regulatory control. Among the various AAV serotypes, AAV2 is most commonly employed.

An AAV vector of the invention typically comprises a polynucleotide that is heterologous to AAV. The polynucleotide is typically of interest because of a capacity to provide a function to a target cell in the context of gene therapy, such as up- or down-regulation of the expression of a certain phenotype. Such a heterologous polynucleotide or “transgene,” generally is of sufficient length to provide the desired function or encoding sequence.

Where transcription of the heterologous polynucleotide is desired in the intended target cell, it can be operably linked to its own or to a heterologous promoter, depending for example on the desired level and/or specificity of transcription within the target cell, as is known in the art. Various types of promoters and enhancers are suitable for use in this context. Constitutive promoters provide an ongoing level of gene transcription, and may be preferred when it is desired that the therapeutic or prophylactic polynucleotide be expressed on an ongoing basis. Inducible promoters generally exhibit low activity in the absence of the inducer, and are up-regulated in the presence of the inducer. They may be preferred when expression is desired only at certain times or at certain locations, or when it is desirable to titrate the level of expression using an inducing agent. Promoters and enhancers may also be tissue-specific: that is, they exhibit their activity only in certain cell types, presumably due to gene regulatory elements found uniquely in those cells.

Illustrative examples of promoters are the SV40 late promoter from simian virus 40, the Baculovirus polyhedron enhancer/promoter element, Herpes Simplex Virus thymidine kinase (HSV tk), the immediate early promoter from cytomegalovirus (CMV) and various retroviral promoters including LTR elements. Inducible promoters include heavy metal ion inducible promoters (such as the mouse mammary tumor virus (mMTV) promoter or various growth hormone promoters), and the promoters from T7 phage which are active in the presence of T7 RNA polymerase. By way of illustration, examples of tissue-specific promoters include various surfactin promoters (for expression in the lung), myosin promoters (for expression in muscle), and albumin promoters (for expression in the liver). A large variety of other promoters are known and generally available in the art, and the sequences of many such promoters are available in sequence databases such as the GenBank database.

Where translation is also desired in the intended target cell, the heterologous polynucleotide will preferably also comprise control elements that facilitate translation (such as a ribosome binding site or “RBS” and a polyadenylation signal). Accordingly, the heterologous polynucleotide generally comprises at least one coding region operatively linked to a suitable promoter, and may also comprise, for example, an operatively linked enhancer, ribosome binding site and poly-A signal. The heterologous polynucleotide may comprise one encoding region, or more than one encoding regions under the control of the same or different promoters. The entire unit, containing a combination of control elements and encoding region, is often referred to as an expression cassette.

The heterologous polynucleotide is integrated by recombinant techniques into or in place of the AAV genomic coding region (i.e., in place of the AAV rep and cap genes), but is generally flanked on either side by AAV inverted terminal repeat (ITR) regions. This means that an ITR appears both upstream and downstream from the coding sequence, either in direct juxtaposition, e.g., (although not necessarily) without any intervening sequence of AAV origin in order to reduce the likelihood of recombination that might regenerate a replication-competent AAV genome. However, a single ITR may be sufficient to carry out the functions normally associated with configurations comprising two ITRs (see, for example, WO 94/13788), and vector constructs with only one ITR can thus be employed in conjunction with the packaging and production methods of the present invention.

The native promoters for rep are self-regulating, and can limit the amount of AAV particles produced. The rep gene can also be operably linked to a heterologous promoter, whether rep is provided as part of the vector construct, or separately. Any heterologous promoter that is not strongly down-regulated by rep gene expression is suitable; but inducible promoters may be preferred because constitutive expression of the rep gene can have a negative impact on the host cell. A large variety of inducible promoters are known in the art; including, by way of illustration, heavy metal ion inducible promoters (such as metallothionein promoters); steroid hormone inducible promoters (such as the MMTV promoter or growth hormone promoters); and promoters such as those from T7 phage which are active in the presence of T7 RNA polymerase. One sub-class of inducible promoters are those that are induced by the helper virus that is used to complement the replication and packaging of the rAAV vector. A number of helper-virus-inducible promoters have also been described, including the adenovirus early gene promoter which is inducible by adenovirus E1A protein; the adenovirus major late promoter; the herpesvirus promoter which is inducible by herpesvirus proteins such as VP16 or ICP4; as well as vaccinia or poxvirus inducible promoters.

Methods for identifying and testing helper-virus-inducible promoters have been described (see, e.g., WO 96/17947). Thus, methods are known in the art to determine whether or not candidate promoters are helper-virus-inducible, and whether or not they will be useful in the generation of high efficiency packaging cells. Briefly, one such method involves replacing the p5 promoter of the AAV rep gene with the putative helper-virus-inducible promoter (either known in the art or identified using well-known techniques such as linkage to promoter-less “reporter” genes). The AAV rep-cap genes (with p5 replaced), e.g., linked to a positive selectable marker such as an antibiotic resistance gene, are then stably integrated into a suitable host cell (such as the HeLa or A549 cells exemplified below). Cells that are able to grow relatively well under selection conditions (e.g., in the presence of the antibiotic) are then tested for their ability to express the rep and cap genes upon addition of a helper virus. As an initial test for rep and/or cap expression, cells can be readily screened using immunofluorescence to detect Rep and/or Cap proteins. Confirmation of packaging capabilities and efficiencies can then be determined by functional tests for replication and packaging of incoming rAAV vectors. Using this methodology, a helper-virus-inducible promoter derived from the mouse metallothionein gene has been identified as a suitable replacement for the p5 promoter, and used for producing high titers of rAAV particles (as described in WO 96/17947).

Removal of one or more AAV genes is in any case desirable, to reduce the likelihood of generating replication-competent AAV (“RCA”). Accordingly, encoding or promoter sequences for rep, cap, or both, may be removed, since the functions provided by these genes can be provided in trans, e.g., in a stable line or via co-transfection.

The resultant vector is referred to as being “defective” in these functions. In order to replicate and package the vector, the missing functions are complemented with a packaging gene, or a plurality thereof, which together encode the necessary functions for the various missing rep and/or cap gene products. The packaging genes or gene cassettes are in one embodiment not flanked by AAV ITRs and in one embodiment do not share any substantial homology with the rAAV genome. Thus, in order to minimize homologous recombination during replication between the vector sequence and separately provided packaging genes, it is desirable to avoid overlap of the two polynucleotide sequences. The level of homology and corresponding frequency of recombination increase with increasing length of homologous sequences and with their level of shared identity. The level of homology that will pose a concern in a given system can be determined theoretically and confirmed experimentally, as is known in the art. Typically, however, recombination can be substantially reduced or eliminated if the overlapping sequence is less than about a 25 nucleotide sequence if it is at least 80% identical over its entire length, or less than about a 50 nucleotide sequence if it is at least 70% identical over its entire length. Of course, even lower levels of homology are preferable since they will further reduce the likelihood of recombination. It appears that, even without any overlapping homology, there is some residual frequency of generating RCA. Even further reductions in the frequency of generating RCA (e.g., by nonhomologous recombination) can be obtained by “splitting” the replication and encapsidation functions of AAV, as described by Allen et al., WO 98/27204).

The rAAV vector construct, and the complementary packaging gene constructs can be implemented in this invention in a number of different forms. Viral particles, plasmids, and stably transformed host cells can all be used to introduce such constructs into the packaging cell, either transiently or stably.

In certain embodiments of this invention, the AAV vector and complementary packaging gene(s), if any, are provided in the form of bacterial plasmids. AAV particles, or any combination thereof. In other embodiments, either the AAV vector sequence, the packaging gene(s), or both, are provided in the form of genetically altered (preferably inheritably altered) eukaryotic cells. The development of host cells inheritably altered to express the AAV vector sequence, AAV packaging genes, or both, provides an established source of the material that is expressed at a reliable level.

A variety of different genetically altered cells can thus be used in the context of this invention. By way of illustration, a mammalian host cell may be used with at least one intact copy of a stably integrated rAAV vector. An AAV packaging plasmid comprising at least an AAV rep gene operably linked to a promoter can be used to supply replication functions (as described in U.S. Pat. No. 5,658,776). Alternatively, a stable mammalian cell line with an AAV rep gene operably linked to a promoter can be used to supply replication functions (see, e.g., Trempe et al., WO 95/13392), Burstein et al. (WO 98/23018); and Johnson et al. (U.S. Pat. No. 5,656,785). The AAV cap gene, providing the encapsidation proteins as described above, can be provided together with an AAV rep gene or separately (see, e.g., the above-referenced applications and patents as well as Allen et al. (WO 98/27204). Other combinations are possible and included within the scope of this invention.

Suitable dose ranges for are generally about 10³ to 10¹⁵ infectious units of viral vector per microliter delivered in 1 to 3000 microliters of single injection volume. For instance, viral genomes or infectious units of vector per micro liter would generally contain about 10⁴, 10⁵, 10⁶, 10⁷, 10⁸, 10⁹, 10¹⁰, 10¹¹, 10¹², 10¹³, 10¹⁴, 10¹⁵, 10¹⁶, or 10¹⁷ viral genomes or infectious units of viral vector delivered in about 10, 50, 100, 200, 500, 1000, or 2000 microliters. It should be understood that the aforementioned dosage is merely an exemplary dosage and those of skill in the art will understand that this dosage may be varied. Effective doses may be extrapolated from dose-responsive curves derived from in vitro or in vivo test systems.

In one embodiment, suitable dose ranges are generally about 10³ to 10¹⁵ infectious units of viral vector per microliter delivered in, for example, 1, 2, 5, 10, 25, 50, 75 or 100 or more milliliters, e.g., 1 to 10,000 milliliters or 0.5 to 15 milliliters, of single injection volume. For instance, viral genomes or infectious units of vector per microliter would generally contain about 10⁴, 10⁵, 10⁶, 10⁷, 10⁸, 10⁹, 10¹⁰, 10¹¹, 10¹², 10¹³, or 10¹⁴ viral genomes or infectious units of viral vector. In one embodiment, suitable dose ranges, generally about 10³ to 10¹⁵ infectious units of viral vector per microliter delivered in, for example, 1, 2, 5, 10, 25, 50, 75 or 100 or more milliliters, e.g., 1 to 10,000 milliliters or 0.5 to 15 milliliters. For instance, viral genomes or infectious units of vector per microliter would generally contain about 10⁴, 10⁵, 10⁶, 10⁷, 10⁸, 10⁹, 10¹⁰, 10¹¹, 10¹², 10¹³, 10¹⁴, 10¹⁵, 10¹⁶, or 10¹⁷ viral genomes or infectious units of viral vector, e.g., at least 1.2×10¹¹ genomes or infectious units, for instance at least 2×10¹¹ up to about 2×10¹² genomes or infectious units or about 1×10¹³ to about 5×10¹⁶ genomes or infectious units.

### Exemplary Nanoparticle Formulations

Biodegradable nanoparticles, e.g., comprising nucleic acid encoding guide-RNA and/or Cas9, or guide-RNA and Cas9, may include or may be formed from biodegradable polymeric molecules which may include, but are not limited to polylactic acid (PLA), polyglycolic acid (PGA), co-polymers of PLA and PGA (i.e., polyactic-co-glycolic acid (PLGA)), poly-ε-caprolactone (PCL), polyethylene glycol (PEG), poly(3-hydroxybutyrate), poly(p-dioxanone), polypropylene fumarate, poly(orthoesters), polyol/diketene acetals addition polymers, poly-alkyl-cyano-acrylates (PAC), poly(sebacic anhydride) (PSA), poly(carboxybiscarboxyphenoxyphenoxy hexone (PCPP) poly[bis (p-carboxypheonoxy)methane](PCPM), copolymers of PSA, PCPP and PCPM, poly(amino acids), poly(pseudo amino acids), polyphosphazenes, derivatives of poly[(dichloro)phosphazenes] and poly[(organo)phosphazenes], poly-hydroxybutyric acid, or S-caproic acid, elastin, or gelatin. (See, e.g., Kumari et al., Colloids and Surfaces B: Biointerfaces 75 (2010) 1-18; and U.S. Pat. Nos. 6,913,767; 6,884,435; 6,565,777; 6,534,092; 6,528,087; 6,379,704; 6,309,569; 6,264,987; 6,210,707; 6,090,925; 6,022,564; 5,981,719; 5,871,747; 5,723,269; 5,603,960; and 5,578,709; and U.S. Published Application No. 2007/0081972; and International Application Publication Nos. WO 2012/115806; and WO 2012/054425; the contents of which are incorporated herein by reference in their entireties).

The biodegradable nanoparticles may be prepared by methods known in the art. (See, e.g., Nagavarma et al., Asian J. of Pharma. And Clin. Res., Vol 5, Suppl 3, 2012, pages 16-23; Cismaru et al., Rev. Roum. Chim., 2010, 55(8), 433-442; and International Application Publication Nos. WO 2012/115806; and WO 2012/054425; the contents of which are incorporated herein by reference in their entireties). Suitable methods for preparing the nanoparticles may include methods that utilize a dispersion of a preformed polymer, which may include but are not limited to solvent evaporation, nanoprecipitation, emulsification/solvent diffusion, salting out, dialysis, and supercritical fluid technology. In some embodiments, the nanoparticles may be prepared by forming a double emulsion (e.g., water-in-oil-in-water) and subsequently performing solvent-evaporation. The nanoparticles obtained by the disclosed methods may be subjected to further processing steps such as washing and lyophilization, as desired. Optionally, the nanoparticles may be combined with a preservative (e.g., trehalose).

Typically, the nanoparticles have a mean effective diameter of less than 1 micron, e.g., the nanoparticles have a mean effective diameter of between about 25 nm and about 500 nm, e.g., between about 50 nm and about 250 nm, about 100 nm to about 150 nm, or about 450 nm to 650 nm. The size of the particles (e.g., mean effective diameter) may be assessed by known methods in the art, which may include but are not limited to transmission electron microscopy (TEM), scanning electron microscopy (SEM), Atomic Force Microscopy (AFM), Photon Correlation Spectroscopy (PCS), Nanoparticle Surface Area Monitor (NSAM), Condensation Particle Counter (CPC), Differential Mobility Analyzer (DMA). Scanning Mobility Particle Sizer (SMPS), Nanoparticle Tracking Analysis (NTA), X-Ray Diffraction (XRD), Aerosol Time of Flight Mass Spectroscopy (ATFMS), and Aerosol Particle Mass Analyzer (APM).

The biodegradable nanoparticles may have a zeta-potential that facilitates uptake by a target cell. Typically, the nanoparticles have a zeta-potential greater than 0. In some embodiments, the nanoparticles have a zeta-potential between about 5 mV to about 45 mV, between about 15 mV to about 35 mV, or between about 20 mV and about 40 mV. Zeta-potential may be determined via characteristics that include electrophoretic mobility or dynamic electrophoretic mobility. Electrokinetic phenomena and electroacoustic phenomena may be utilized to calculate zeta-potential.

In one embodiment, a non-viral delivery vehicle comprises polymers including but not limited to poly(lactic-co-glycolic acid) (PLGA), polylactic acid (PLA), linear and/or branched PEI with differing molecular weights (e.g., 2, 22 and 25 kDa), dendrimers such as polyamidoamine (PAMAM) and polymethoacrylates; lipids including but not limited to cationic liposomes, cationic emulsions, DOTAP, DOTMA, DMRIE, DOSPA, distearoylphosphatidylcholine (DSPC), DOPE, or DC-cholesterol; peptide based vectors including but not limited to Poly-L-lysine or protamine; or poly(β-amino ester), chitosan, PEI-polyethylene glycol, PEI-mannose-dextrose, DOTAP-cholesterol or RNAiMAX.

In one embodiment, the delivery vehicle is a glycopolymer-based delivery vehicle, poly(glycoamidoamine)s (PGAAs), that have the ability to complex with various polynucleotide types and form nanoparticles. These materials are created by polymerizing the methylester or lactone derivatives of various carbohydrates (D-glucarate (D), meso-galactarate (G), D-mannarate (M), and L-tartarate (T)) with a series of oligoethyleneamine monomers (containing between 1-4 ethylenamines (Liu and Reineke, 2006). A subset composed of these carbohydrates and four ethyleneamines in the polymer repeat units yielded exceptional delivery efficiency.

In one embodiment, the delivery vehicle comprises polyethyleneimine (PEI), Polyamidoamine (PAMAM), PEI-PEG, PEI-PEG-mannose, dextran-PEI, OVA conjugate, PLGA microparticles, or PLGA microparticles coated with PAMAM, or any combination thereof. The disclosed cationic polymer may include, but are not limited to, polyamidoamine (PAMAM) dendrimers. Polyamidoanine dendrimers suitable for preparing the presently disclosed nanoparticles may include 3rd-, 4th-, 5th-, or at least 6th-generation dendrimers.

In one embodiment, the delivery vehicle comprises a lipid, e.g., N-[1-(2,3-dioleoyloxy)propel]-N,N,N-trimethylammonium (DOTMA), 2,3-dioleyloxy-N-[2-spermine carboxamidel ethyl-N,N-dimethyl-1-propanammonium trifluoracetate (DOSPA, Lipofectamine); 1,2-dioleoyl-3-trimethylammonium-propane (DOTAP); N-[1-(2,3-dimyristloxy) propyl]; N,N-dimethyl-N-(2-hydroxyethyl) ammonium bromide (DMRIE), 3-β-[N—(N,N-dimethylaminoethane) carbamoyl] cholesterol (DC-Chol); dioctadecyl amidoglyceryl spermine (DOGS, Transfectam); or imethyldioctadeclyammonium bromide (DDAB). The positively charged hydrophilic head group of cationic lipids usually consists of monoamine such as tertiary and quaternary amines, polyamine, amidinium, or guanidinium group. A series of pyridinium lipids have been developed (Zhu et al., 2008; van der Woude et al., 1997; Ilies et al., 2004). In addition to pyridinium cationic lipids, other types of heterocyclic head group include imidazole, piperizine and amino acid. The main function of cationic head groups is to condense negatively charged nucleic acids by means of electrostatic interaction to slightly positively charged nanoparticles, leading to enhanced cellular uptake and endosomal escape.

Lipids having two linear fatty acid chains, such as DOTMA, DOTAP and SAINT-2, or DODAC, may be employed as a delivery vehicle, as well as tetraalkyl lipid chain surfactant, the dimer of N,N-dioleyl-N,N-dimethylammonium chloride (DODAC). All the trans-orientated lipids regardless of their hydrophobic chain lengths (C_{16:1}, C_{18:1} and C_{20:1}) appear to enhance the transfection efficiency compared with their cis-orientated counterparts.

The structures of cationic polymers useful as a delivery vehicle include but are not limited to linear polymers such as chitosan and linear poly(ethyleneimine), branched polymers such as branch poly(ethyleneimine) (PEI), circle-like polymers such as cyclodextrin, network (crosslinked) type polymers such as crosslinked poly(amino acid) (PAA), and dendrimers. Dendrimers consist of a central core molecule, from which several highly branched arms ‘grow’ to form a tree-like structure with a manner of symmetry or asymmetry. Examples of dendrimers include polyamidoamine (PAMAM) and polypropylenimine (PPI) dendrimers.

DOPE and cholesterol are commonly used neutral co-lipids for preparing cationic liposomes. Branched PEI-cholesterol water-soluble lipopolymer conjugates self-assemble into cationic micelles. Pluronic (poloxamer), anon-ionic polymer and SP1017, which is the combination of Pluronics L61 and F127, may also be used.

In one embodiment, PLGA particles are employed to increase the encapsulation frequency although complex formation with PLL may also increase the encapsulation efficiency. Other cationic materials, for example, PEI, DOTMA, DC-Chol, or CTAB, may be used to make nanospheres.

In one embodiment, complexes are embedded in or applied to a material including but not limited to hydrogels of poloxamers, polyacrylamide, poly(2-hydroxyethyl methacrylate), carboxyvinyl-polymers (e.g., Carbopol 934, Goodrich Chemical Co.), cellulose derivatives, e.g., methylcellulose, cellulose acetate and hydroxypropyl cellulose, polyvinyl pyrrolidone or polyvinyl alcohols, or combinations thereof.

In some embodiments, a biocompatible polymeric material is derived from a biodegradable polymeric such as collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N-isopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), or poly(dioxanone) (PPS).

In another embodiment, the biocompatible material includes polyethyleneterephalate, polytetrafluoroethylene, copolymer of polyethylene oxide and polypropylene oxide, a combination of polyglycolic acid and polyhydroxyalkanoate, gelatin, alginate, poly-3-hydroxybutyrate, poly-4-hydroxybutyrate, and polyhydroxyoctanoate, and polyacrylonitrilepolyvinylchlorides.

In one embodiment, the following polymers may be employed, e.g., natural polymers such as starch, chitin, glycosaminoglycans, e.g., hyaluronic acid, dermatan sulfate and chrondrotin sulfate, and microbial polyesters, e.g., hydroxyalkanoates such as hydroxyvalerate and hydroxybutyrate copolymers, and synthetic polymers. e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol(D,L-lactide-coglycolide), poly(lactic acid colysine) and polycaprolactone.

In one embodiment, the biocompatible material is derived from isolated extracellular matrix (ECM). ECM may be isolated from endothelial layers of various cell populations, tissues and/or organs, e.g., any organ or tissue source including the dermis of the skin, liver, alimentary, respiratory, intestinal, urinary or genital tracks of a warm blooded vertebrate. ECM employed in the invention may be from a combination of sources. Isolated ECM may be prepared as a sheet, in particulate form, gel form and the like.

The biocompatible scaffold polymer may comprise silk, elastin, chitin, chitosan, poly(d-hydroxy acid), poly(anhydrides), or poly(orthoesters). More particularly, the biocompatible polymer may be formed polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy) methane] (PCPM), copolymers of SA, CPP and CPM, poly(amino acids), poly(pseudo amino acids), polyphosphazenes, derivatives of poly[(dichloro)phosphazenes] or poly[(organo) phosphazenes], poly-hydroxybutyric acid, or S-caproic acid, polylactide-co-glycolide, polylactic acid, polyethylene glycol, cellulose, oxidized cellulose, alginate, gelatin or derivatives thereof.

Thus, the polymer may be formed of any of a wide range materials including polymers, including naturally occurring polymers, synthetic polymers, or a combination thereof. In one embodiment, the scaffold comprises biodegradable polymers. In one embodiment, a naturally occurring biodegradable polymer may be modified to provide for a synthetic biodegradable polymer derived from the naturally occurring polymer. In one embodiment, the polymer is a poly(lactic acid) (“PLA”) or poly(lactic-co-glycolic acid) (“PLGA”). In one embodiment, the scaffold polymer includes but is not limited to alginate, chitosan, poly(2-hydroxyethylmethacrylate), xyloglucan, co-polymers of 2-methacryloyloxyethyl phosphorylcholine, poly(vinyl alcohol), silicone, hydrophobic polyesters and hydrophilic polyester, poly(lactide-co-glycolide), N-isoproylacrylamide copolymers, poly(ethylene oxide)/poly(propylene oxide), polylactic acid, poly(orthoesters), polyanhydrides, polyurethanes, copolymers of 2-hydroxyethylmethacrylate and sodium methacrylate, phosphorylcholine, cyclodextrins, polysulfone and polyvinylpyrrolidine, starch, poly-D,L-lactic acid-para-dioxanone-polyethylene glycol block copolymer, polypropylene, poly(ethylene terephthalate), poly(tetrafluoroethylene), poly-epsilon-caprolactone, or crosslinked chitosan hydrogels.

### Pharmaceutical Compositions

The disclosure provides a composition comprising, consisting essentially of, or consisting of the above-described gene transfer vector(s) and a pharmaceutically acceptable (e.g., physiologically acceptable) carrier. In one embodiment, when the composition consists essentially of the gene transfer vector complexed with a cationic polymer or encapsulated in nanoparticles, or is a recombinant virus, and a pharmaceutically acceptable carrier, additional components can be included that do not materially affect the composition (e.g., adjuvants, buffers, stabilizers, anti-inflammatory agents, solubilizers, preservatives, etc.). In one embodiment, when the composition consists of the gene transfer vector complexed with the cationic polymer or encapsulated in nanoparticles, or is a recombinant virus, and the pharmaceutically acceptable carrier, the composition does not comprise any additional components. Any suitable carrier can be used within the context of the invention, and such carriers are well known in the art. The choice of carrier will be determined, in part, by the particular site to which the composition may be administered and the particular method used to administer the composition. The composition optionally can be sterile with the exception of, in one embodiment, the gene transfer vector complexed with the cationic polymer or encapsulated in nanoparticles, or recombinant virus, described herein. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, PA (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. In one embodiment, the carrier is a buffered saline solution. In one embodiment, the gene transfer vector is administered in a composition formulated to protect the gene transfer vector from damage prior to administration. For example, the composition can be formulated to reduce loss of the gene transfer vector on devices used to prepare, store, or administer the gene transfer vector, such as glassware, syringes, or needles. The composition can be formulated to decrease the light sensitivity and/or temperature sensitivity of the gene transfer vector. To this end, the composition may comprise a pharmaceutically acceptable liquid carrier, such as, for example, those described above, and a stabilizing agent selected from the group consisting of polysorbate 80, L-arginine, pol vinylpyrrolidone, trehalose, and combinations thereof. Use of such a composition will extend the shelf life of the gene transfer vector, facilitate administration, and increase the efficiency of the inventive method. Formulations for gene transfer vector containing compositions are further described in, for example, Wright et al., Curr. Opin. Drug Discov. Devel., 6(2): 174-178 (2003) and Wright et al., Molecular Therapy, 12: 171-178 (2005)) The composition also can be formulated to enhance transduction efficiency. In addition, one of ordinary skill in the art will appreciate that the gene transfer vector can be present in a composition with other therapeutic or biologically-active agents. For example, factors that control inflammation, such as ibuprofen or steroids, can be part of the composition to reduce swelling and inflammation associated with in vivo administration of the gene transfer vector. Immune system stimulators or adjuvants, e.g., interleukins, lipopolysaccharide, and double-stranded RNA. Antibiotics, i.e., microbicides and fungicides, can be present to treat existing infection and/or reduce the risk of future infection, such as infection associated with gene transfer procedures.

Injectable depot forms are made by forming microencapsulated matrices of the subject vectors in biodegradable polymers such as polylactide-polyglycolide. Depending on the ratio of vector to polymer, and the nature of the particular polymer employed, the rate of vector release can be controlled. Examples of other biodegradable polymers include poly(orthoesters) and poly(anhydrides). Depot injectable formulations are also prepared by entrapping the vector optionally in a complex with a cationic polymer in liposomes or microemulsions which are compatible with body tissue.

In certain embodiments, a formulation comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see. e.g., U.S. Pat. No. 4,863,457), such as an implantable device, e.g., a mechanical reservoir or an implant or a device comprised of a polymeric composition, are particularly useful for administration of the inventive gene transfer vector. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

The dose of the gene transfer vector in the composition administered to the mammal will depend on a number of factors, including the size (mass) of the mammal, the extent of any side-effects, the particular route of administration, and the like. In one embodiment, the inventive method comprises administering a “therapeutically effective amount” of the composition comprising the inventive gene transfer vector described herein. A “therapeutically effective amount” refers to an amount effective, at dosages and for periods of time necessary, to achieve a desired therapeutic result. The therapeutically effective amount may vary according to factors such as the extent of the disease or disorder, age, sex, and weight of the individual, and the ability of the gene transfer vector to elicit a desired response in the individual. The dose of gene transfer vector in the composition required to achieve a particular therapeutic effect typically is administered in units of vector genome copies per cell (gc/cell) or vector genome copies/per kilogram of body weight (gc/kg). One of ordinary skill in the art can readily determine an appropriate gene transfer vector dose range to treat a patient having a particular disease or disorder, based on these and other factors that are well known in the art. In one embodiment, the therapeutically effective amount may be between 1×10¹⁰ genome copies to 1×10¹³ genome copies for viruses.

In one embodiment, the composition is administered once to the mammal. It is believed that a single administration of the composition may result in persistent expression in the mammal with minimal side effects. However, in certain cases, it may be appropriate to administer the composition multiple times during a therapeutic period to ensure sufficient exposure of cells to the composition. For example, the composition may be administered to the mammal two or more times (e.g., 2, 3, 4, 5, 6, 6, 8, 9, or 10 or more times) during a therapeutic period.

The present disclosure provides pharmaceutically acceptable compositions which comprise a therapeutically-effective amount of gene transfer vector comprising a nucleic acid sequence as described above.

### Routes of Administration, Dosages and Dosage Forms

Administration of the gene delivery vectors may be continuous or intermittent, depending, for example, upon the recipient's physiological condition, and other factors known to skilled practitioners. The administration of the gene delivery vector(s) may be essentially continuous over a preselected period of time or may be in a series of spaced doses. Both local administration, e.g., to the eye, intranasal or intrathecal, and systemic administration are contemplated. Any route of administration may be employed, e.g., intravenous, intranasal or to the eye.

One or more suitable unit dosage forms comprising the gene delivery vector(s), which may optionally be formulated for sustained release, can be administered by a variety of routes including local, oral, or parenteral, including by rectal, buccal, vaginal and sublingual, transdermal, subcutaneous, intravenous, intramuscular, intraperitoneal, intrathoracic, or intrapulmonary routes. The formulations may, where appropriate, be conveniently presented in discrete unit dosage forms and may be prepared by any of the methods well known to pharmacy. Such methods may include the step of bringing into association the vector with liquid carriers, solid matrices, semi-solid carriers, finely divided solid carriers or combinations thereof, and then, if necessary, introducing or shaping the product into the desired delivery system.

The amount of gene delivery vector(s) administered to achieve a particular outcome will vary depending on various factors including, but not limited to, the genes and promoters chosen, the condition, patient specific parameters, e.g., height, weight and age, and whether prevention or treatment, is to be achieved.

Vectors of the invention may conveniently be provided in the form of formulations suitable for administration. A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. By “pharmaceutically acceptable” it is meant a carrier, diluent, excipient, and/or salt that is compatible with the other ingredients of the formulation, and not deleterious to the recipient thereof.

Vectors of the present invention may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Therapeutically useful compositions can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The vectors can be provided in a dosage form containing an amount of a vector effective in one or multiple doses. For viral vectors, the effective dose may be in the range of at least about 10⁷ viral particles, e.g., about 10⁹ viral particles, or about 10¹¹ viral particles. The number of viral particles added may be up to 10¹⁴. For example, when a viral expression vector is employed, about 10⁸ to about 10⁶⁰ gc of viral vector can be administered as nucleic acid or as a packaged virion. In some embodiments, about 10⁹ to about 10⁵ copies of viral vector, e.g., per 0.5 to 10 mL, can be administered as nucleic acid or as a packaged virion. Alternatively, the nucleic acids or vectors, can be administered in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results. The amount administered will vary depending on various factors including, but not limited to, the nucleic acid or vector chosen for administration, the disease, the weight, the physical condition, the health, and/or the age of the mammal. Such factors can be readily determined by the clinician employing animal models or other test systems that are available in the art. As noted, the exact dose to be administered is determined by the attending clinician but may be in 1 mL phosphate buffered saline. For delivery of plasmid DNA alone, or plasmid DNA in a complex with other macromolecules, the amount of DNA to be administered will be an amount which results in a beneficial effect to the recipient. For example, from 0.0001 to 1 mg or more, e.g., up to 1 g, in individual or divided doses, e.g., from 0.001 to 0.5 mg, or 0.01 to 0.1 mg, of DNA can be administered.

For example, when a viral expression vector is employed, about 10⁸ to about 10⁶⁰ gc of viral vector can be administered as nucleic acid or as a packaged virion. In some embodiments, about 10⁹ to about 10¹⁵ copies of viral vector, e.g., per 0.5 to 10 mL, can be administered as nucleic acid or as a packaged virion. Alternatively, the nucleic acids or vectors, can be administered in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results.

By way of illustration, liposomes and other lipid-containing gene delivery complexes can be used to deliver one or more transgenes. The principles of the preparation and use of such complexes for gene delivery have been described in the art (see, e.g., Ledley, (1995); Miller et al., (1995); Chonn et al., (1995); Schofield et al., (1995); Brigham et al., (1993)).

Pharmaceutical formulations containing the gene delivery vectors can be prepared by procedures known in the art using well known and readily available ingredients. For example, the agent can be formulated with common excipients, diluents, or carriers, and formed into tablets, capsules, suspensions, powders, and the like. The vectors of the invention can also be formulated as elixirs or solutions appropriate for parenteral administration, for instance, by intramuscular, subcutaneous or intravenous routes.

The pharmaceutical formulations of the vectors can also take the form of an aqueous or anhydrous solution, e.g., a lyophilized formulation, or dispersion, or alternatively the form of an emulsion or suspension.

In one embodiment, the vectors may be formulated for administration. e.g., by injection, for example, bolus injection or continuous infusion via a catheter, and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

For administration to the upper (nasal) or lower respiratory tract by inhalation, the vector is conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the vector may be administered via nose drops, a liquid spray, such as via a plastic bottle atomizer or metered-dose inhaler. Typical of atomizers are the Mistometer (Wintrop) and the Medihaler (Riker).

The local delivery of the vectors can also be by a variety of techniques which administer the vector at or near the site of disease, e.g., using a catheter or needle. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

The formulations and compositions described herein may also contain other ingredients such as antimicrobial agents or preservatives.

### Subjects

The subject may be any animal, including a human and non-human animals. Non-human animals includes all vertebrates, e.g., mammals and non-mammals, such as non-human primates, sheep, dogs, pigs, cats, cows, horses, chickens, amphibians, and reptiles, although in one embodiment mammals are preferred, such as non-human primates, sheep, pigs, dogs, cats, cows and horses. The subject may also be livestock such as, cattle, swine, sheep, poultry, and horses, or pets, such as dogs and cats.

Subjects include human subjects suffering from or at risk for oxidative damage. The subject is generally diagnosed with the condition of the subject invention by skilled artisans, such as a medical practitioner.

The methods described herein can be employed for subjects of any species, gender, age, ethnic population, or genotype. Accordingly, the term subject includes males and females, and it includes elderly, elderly-to-adult transition age subjects adults, adult-to-pre-adult transition age subjects, and pre-adults, including adolescents, children, and infants.

Examples of human ethnic populations include Caucasians. Asians. Hispanics, Africans, African Americans, Native Americans, Semites, and Pacific Islanders. The methods of the invention may be more appropriate for some ethnic populations such as Caucasians, especially northern European populations, as well as Asian populations.

The term subject also includes subjects of any genotype or phenotype as long as they are in need of the invention, as described above. In addition, the subject can have the genotype or phenotype for any hair color, eye color, skin color or any combination thereof.

The term subject includes a subject of any body height, body weight, or any organ or body part size or shape.

### Exemplary Myocilin Sequences

An exemplary mRNA sequence for human myocilin is:

(SEQ ID NO: 4)
cgggccccgg acacccgctc tgcacagcag agctttccag aggaagcctc accaagcctc
tgcaatgagg ttcttctgtg cacgttgctg cagctttggg cctgagatgc cagctgtcca
gctgctgctt ctggcctgcc tggtgtggga tgtgggggcc aggacagctc agctcaggaa
ggccaatgac cagagtggcc gatgccagta taccttcagt gtggccagtc ccaatgaatc
cagctgccca gagcagagcc aggccatgtc agtcatccat aacttacaga gagacagcag
cacccaacgc ttagacctgg aggccaccaa agctcgactc agctccctgg agagcctcct
ccaccaattg accttggacc aggctgccag gccccaggag acccaggagg ggctgcagag
ggagctgggc accctgaggc gggagcggga ccagctggaa acccaaacca gagagttgga
gactgcctac agcaacctcc tccgagacaa gtcagttctg gaggaagaga agaagcgact
aaggcaagaa aatgagaatc tggccaggag gttggaaagc agcagccagg aggtagcaag
gctgagaagg ggccagtgtc cccagacccg agacactgct cgggctgtgc caccaggctc
cagagaagtt tctacgtgga atttggacac tttggccttc caggaactga agtccgagct
aactgaagtt cctgcttccc gaattttgaa ggagagccca tctggctatc tcaggagtgg
agagggagac accggatgtg gagaactagt ttgggtagga gagcctctca cgctgagaac
agcagaaaca attactggca agtatggtgt gtggatgcga gaccccaagc ccacctaccc
ctacacccag gagaccacgt ggagaatcga cacagttggc acggatgtcc gccaggtttt
tgagtatgac ctcatcagcc agtttatgca gggctaccct tctaaggttc acatactgcc
taggccactg gaaagcacgg gtgctgtggt gtactcgggg agcctctatt tccagggcgc
tgagtccaga actgtcataa gatatgagct gaataccgag acagtgaagg ctgagaagga
aatccctgga gctggctacc acggacagtt cccgtattct tggggtggct acacggacat
tgacttggct gtggatgaag caggcctctg ggtcatttac agcaccgatg aggccaaagg
tgccattgtc ctctccaaac tgaacccaga gaatctggaa ctcgaacaaa cctgggagac
aaacatccgt aagcagtcag tcgccaatgc cttcatcatc tgtggcacct tgtacaccgt
cagcagctac acctcagcag atgctaccgt caactttgct tatgacacag gcacaggtat
cagcaagacc ctgaccatcc cattcaagaa ccgctataag tacagcagca tgattgacta
caaccccctg gagaagaagc tctttgcctg ggacaacttg aacatggtca cttatgacat
caagctctcc aagatgtgaa aagcctccaa gctgtacagg caatggcaga aggagatgct
cagggctcct ggggggagca ggctgaaggg agagccagcc agccagggcc caggcagctt
tgactgcttt ccaagttttc attaatccag aaggatgaac atggtcacca tctaactatt
caggaattgt agtctgaggg cgtagacaat ttcatataat aastatcctt tatcttctgt
cagcatttat gggatgttta atgacatagt tcaagttttc tigtgatttg gggcaaaagc
tgtaaggcat aatagtttct tcctgaaaac cattgctctt gcatgttaca tgggtaccac
aagccacaat aaaa.

Exemplary guide-RNA sequences may be the same as or the complementary sequence to about 20 to 25 bases in SEQ ID NO:4, or a sequence with one, two or three nucleotide substitutions, e.g., guide-RNA sequences may be the same as or the complementary sequence to about 20 to 25 bases in SEQ ID NO:1 or 2, or a sequence with one, two or three nucleotide substitutions.

An exemplary human myocilin sequence is:

(SEQ ID NO: 5)
MRFFCARCCSFGPEMPAVQLLLLACLVWDVGARTAQLRKANDQS
GRCQYTFSVASPNESSCPEQSQAMSVIHNLQRDSSTQRLDLEATKARLS
SLESLLHQLTLDQAARPQETQEGLQRELGTLRRERDQLETQTRELETAY
SNLLRDKSVLEEEKKRLRQENENLARRLESSSQEVARLRRGQCPQTRDT
ARAVPPGSREVSTWNLDTLAFQELKSELTEVPASRILKESPSGYLRSGE
GDTGCGELVWVGEPLTLRTAETITGKYGVWMRDPKPTYPYTQETTWRID
TVGTDVRQVFEYDLISQFMQGYPSKVHILPRPLESTGAVVYSGSLYFQG
AESRTVIRYELNTETVKAEKEIPGAGYHGQFPYSWGGYTDIDLAVDEAG
LWVIYSTDEAKGAIVLSKLNPENLELEQTWETNIRKQSVANAFIICGTL
YTVSSYTSADATVNFAYDTGTGISKTLTIPFKNRYKYSSMIDYNPLEKK
LFAWDNLNMVTYDIKLSKM

In one embodiment, the mutation introduced using the gRNA and Cas results in a truncated myocilin, e.g., one that is truncated at the C-terminus by at least 50, 100, 250, 200, 250, 300, 350, 400 or 450 amino acids, that does not result in an unfolded protein response. In one embodiment, the mutation introduced using the gRNA and Cas results in a truncated myocilin, e.g., one that is truncated at the C-terminus by at least 150, 250, 200, 250, 300, 350, 400 or 450 amino acids, that does not result in an unfolded protein response.

In one embodiment, the mutation introduced using the gRNA and Cas results in a truncated myocilin which is truncated upstream of exon 3, e.g., at a residue before residue 368, in exon 2 or in exon 1, that does not result in an unfolded protein response.

In one embodiment, the mutation introduced using the gRNA and Cas results in a mutation in the peroxisome targeting sequence, e.g., SKM, that does not result in an unfolded protein response.

A partial genomic myocilin sequence showing the position of guide-RNAs having SEQ ID NO:1 and SEQ ID NO:2 is as follows:

gatctccagt tcctagcata gtgcctggca cagtgcaggt tctcaatgag tttgcagagt
gaatggaaat ataaactaga aatatatcct tgttgaaatc agcacaccag tagtcctggt
gtaagtgtgt gtacgtgtgt gtgtgtgtgt gtgtgtgtgt aaaaccaggt ggagatatag
gaactattat tggggtatgg gtgcataaat tgggatgttc tttttaaaaa gaaactccaa
acagacttcc ggaaggttat tttctaagaa tcttgctggc agcgtgaagg caacccccct
gtgcacagcc ccacccagcc tcacgtggcc acctctgtct tcccccatga agggctggct
ccccagtata tataaacctc tctggagctc gggcatgagc cagcaaggcc acccatccag
gcacctctca gcacagcaga gctttccaga ggaagcctca ccaagcctct gcaatgaggt
tcttctgtgc acgttgctgc agctttgggc ctgagatgcc agctgtccag ctgctgcttc
tggcctgcct ggtgtgggat gtgggggcca ggacagctca gctcaggaag gccaatgacc
agagtggccg atgccagtat accttcagtg tggccagtcc caatgaatcc agctgcccag
agcagagcca ggccatgtca gtcatccata acttacagag agacagcagc acccaacgct
tagacctgga ggccaccaaa gctcgactca gctccctgga gagcctcctc caccaattga
ccttggacca ggctgccagg ccccaggaga cccaggaggg gctgcagagg gagctgggca
ccctgaggcg ggagcgggac cagctggaaa cccaaaccag agagttggag actgcctaca
gcaacctcct ccgagacaag tcagttctgg aggaagagaa gaagcgacta aggcaagaaa
atgagaatct ggccaggagg ttggaaagca gcagccagga ggtagcaagg ctgagaaggg
gccagtgtcc ccagacccga gacactgctc gggctgtgcc accaggctcc agagaaggta
agaatgcaga gtggggggac tctgagttca gcaggtgata tggctcgtag tgacctgcta
caggcgctcc aggcctccct gcctgccctt tctcctagag actgcacagc tagcacaaga
cagatgaatt aaggaaagca cagcgatc

### Exemplary Embodiments

In one embodiment, an isolated nucleic acid vector is provided comprising a first expression cassette comprising a first promoter operably linked to an open reading frame for a Cas recombinase and a second expression cassette comprising a second promoter operably linked to a nucleotide sequence comprising a myocilin specific gRNA, wherein the vector is not an adenoviral vector. In one embodiment, the vector is a viral vector, e.g., an adeno-associated virus (AAV), retrovirus or lentivirus vector. In one embodiment, the Cas is saCas9. In one embodiment, the gRNA comprises SEQ ID NO:1 or a nucleotide sequence with at least 90% nucleotide sequence identity thereto. In one embodiment, the gRNA comprises SEQ ID NO:2 or a nucleotide sequence with at least 90% nucleotide sequence identity thereto. In one embodiment, the first promoter is a heterologous RNA polymerase I promoter, e.g., a CMV promoter or a myocilin promoter. In one embodiment, the second promoter is a RNA polymerase III promoter, e.g., an U6 promoter. In one embodiment, the vector of is in a nanoparticle, e.g., a liposome.

In one embodiment, an isolated nucleic acid vector comprising a first expression cassette comprising a first promoter operably linked to an open reading frame for a Cas recombinase and a second expression cassette comprising a second promoter operably linked to a nucleotide sequence comprising a gRNA having SEQ ID NO:1 or SEQ ID NO:2 or a nucleotide sequence with at least 90% nucleotide sequence identity thereto. In one embodiment, the vector is a viral vector, e.g., an adeno-associated virus (AAV), retrovirus or lentivirus vector. In one embodiment, the Cas is saCas9. In one embodiment, the gRNA comprises SEQ ID NO:1 or a nucleotide sequence with at least 90% nucleotide sequence identity thereto. In one embodiment, the gRNA comprises SEQ ID NO:2 or a nucleotide sequence with at least 90% nucleotide sequence identity thereto. In one embodiment, the first promoter is a heterologous RNA polymerase I promoter, e.g., a CMV promoter or a myocilin promoter. In one embodiment, the second promoter is a RNA polymerase III promoter, e.g., an U6 promoter. In one embodiment, the vector of is in a nanoparticle, e.g., a liposome.

In one embodiment, a method to prevent, inhibit or treat glaucoma in a mammal is provided comprising: administering to the mammal an effective amount of a composition having the vector. In one embodiment, the mammal is a human. In one embodiment, the composition is injected into the anterior segment of the eye. In one embodiment, the composition is administered to trabecular meshwork cells. In one embodiment, the composition is intravitreally administered. In one embodiment, the composition is intracamerally administered. In one embodiment, the administration results inactivation of at least one myocilin gene. In one embodiment, the administration results in expression of a truncated myocilin. In one embodiment, the vector is a viral vector. In one embodiment, the viral vector is an adeno-associated viral vector. In one embodiment, the capsid is an AAV2, AAV5 or AAV9 capsid. In one embodiment, the genome is a rAAV2 genome. In one embodiment, the composition is a liposome comprising the vector. In one embodiment, the mammal has primary open angle glaucoma.

In one embodiment, a method to decrease intraocular pressure in a mammal is provided comprising: administering to the mammal an effective amount of a composition having the vector. In one embodiment, the mammal is a human. In one embodiment, the composition is injected into the anterior segment of the eye. In one embodiment, the composition is administered to trabecular meshwork cells. In one embodiment, the composition is intravitreally administered. In one embodiment, the composition is intracamerally administered. In one embodiment, the administration results inactivation of at least one myocilin gene. In one embodiment, the administration results in expression of a truncated myocilin. In one embodiment, the vector is a viral vector. In one embodiment, the viral vector is an adeno-associated viral vector. In one embodiment, the capsid is an AAV2, AAV5 or AAV9 capsid. In one embodiment, the genome is a rAAV2 genome. In one embodiment, the composition is a liposome comprising the vector. In one embodiment, the mammal has primary open angle glaucoma.

In one embodiment, a method to reduce MYOC expression in a mammal is provided comprising: administering to the mammal an effective amount of a composition having the vector. In one embodiment, the mammal is a human. In one embodiment, the composition is injected into the anterior segment of the eye. In one embodiment, the composition is administered to trabecular meshwork cells. In one embodiment, is intracamerally administered. In one embodiment, the administration results inactivation of at least one myocilin gene. In one embodiment, the administration results in expression of a truncated myocilin. In one embodiment, the vector is a viral vector. In one embodiment, the viral vector is an adeno-associated viral vector. In one embodiment, the capsid is an AAV2, AAV5 or AAV9 capsid. In one embodiment, the genome is a rAAV2 genome. In one embodiment, the composition is a liposome comprising the vector. In one embodiment, the mammal has primary open angle glaucoma.

The invention will be described by the following non-limiting examples.

### Example I

### Exemplary Gene Editing Systems and Target Cells

The disclosure provides for, in one embodiment, CRISPRCas9 genome editing of a major glaucoma pathogenesis pathway in the TM responsible for increased AH outflow resistance and elevated TOP. This approach provides a disease modifying therapy compared to current therapies that do not address underlying glaucoma pathogenesis. In one embodiment, AAV delivery to the TM is used to knockout key genes responsible for glaucomatous damage. This single curative therapy eliminates current therapeutic disadvantages including patient noncompliance to pharmaceutical therapies. The efficacy of the AAV.CRISPR-Cas9 system is evaluated in human TM cells, mouse models of POAG and ex vivo in human POC anterior segments.

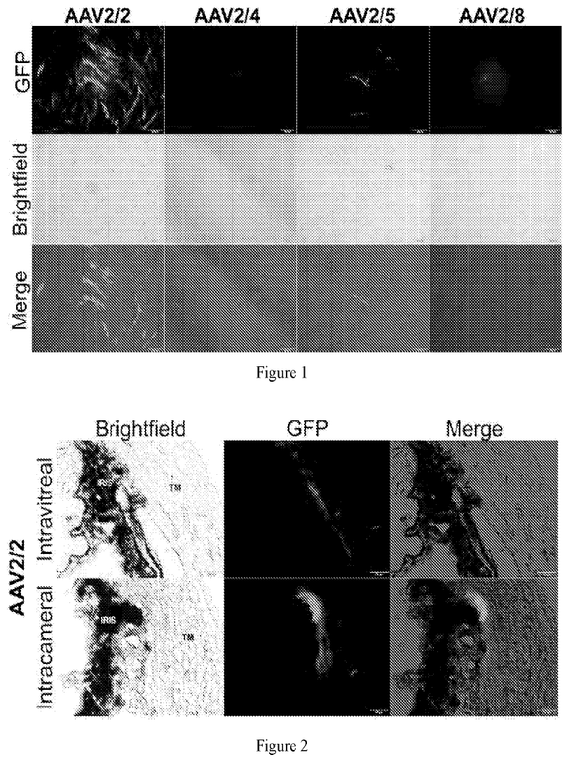

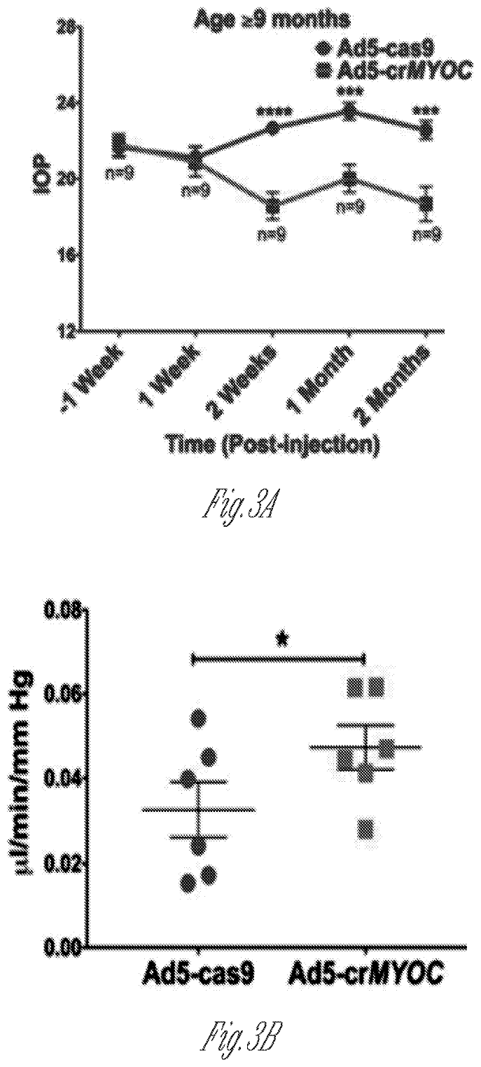

In one embodiment, AAV is used as CRISPR-Cas9 delivery vectors because of the well established safety and effectiveness in several ocular clinical studies (Boye et al., 2013; Acland et al., 2005; Bainbridge et al., 2015; Jacobson et al., 2012). In addition, recent studies (Wang et al., 2017) and data (FIGS. 1 and 2) further support that selected AAVs can express the gene of interest in TM cells efficiently without having significant ocular toxicity. AAV serotypes that provide sufficient transduction of the TM are determined. It is also determined whether scDNA is required for adequate TM vector expression (Wang et al., 2017; Bogner et al., 2015; Borras et al., 2006; Hickey et al., 2017).

### Data

Transduction of AAV2.GFP AAV4.GFP AAV5.GFP and AAV9.GFP in cultured human TM cells. Several studies have shown that AAVs have selected tropism for different cell types (Hickey et al., 2017). Previous studies have shown that only selected AAVs are able to transduce TM cells in mice, rats and monkeys (Wang et al., 2017; Bogner et al., 2015; Hickey et al., 2017). Although previous studies suggested that self-complementary (Sc) AAVs are required to transduce TM cells (48, 49), a recent study has shown that single stranded AAVs can efficiently transduce TM cells (Wang et al., 2017). To clarify which AAVs can transduce TM, GFP labelled AAVs of various serotypes were used to transduce human primary TM cells (FIG. 1). Human primary TM cells (n=2 strains) were grown to confluency in 12-well plates and treated with AAV2/2, AAV2/4, AAV2/5 and AAV2/8 at multiplicities of infection (MOI) of 2.5×10¹ to 2.5×10³ viral genomes (VG)/cell (Wang et al., 2017). GFP expression was examined by fluorescent microscopy after 72 hours of transduction. No AAVs caused GFP expression at MOI of 2.5×10¹ VG/cell (not shown), but robust AAV2-GFP expression was observed at MOI of 2.5×10³ VG/cell. Note that these high MOI are consistent with other cell types (Wang et al., 2017; Bogner et al., 2015; Ellis et al., 2013).

AAV2-GFP efficiently transduces TM cells in C57 mice. Next it was examined whether AAV2 and AAV5 (selected based on the above results) express GFP in mouse TM (FIG. 2). Anesthetized mice were injected with AAV2/2-GFP (2×10⁹ VG/eye) or AAV2/5-GFP (2×10⁹ VG/eye) in one eye via intravitreal (top panel) injection, while the contralateral eyes were injected with same virus intracamerally. After 2-weeks, GFP expression was observed by fluorescent microscopy. Both intravitreal and intracameral routes of administration of AAV2/2-GFP show expression of GFP in TM, although the expression is stronger in intracameral injected eyes (bottom panel). Consistent with primary human TM cells, AAV2/5-GFP did not transduce the TM by these routes of administration.

### Experimental Design

Determine the most efficient AAV vector(s) for transducing cultured human and mouse TM cells. To accomplish this, the tropism of nine different natural AAV serotypes (serotypes 1-9) was determined, including ssAAV (single-strand AAV) and scAAV (self-complementary AAV) vectors that express eGFP driven by the CMV promoter. Even though ssAAV has a larger cloning capacity than scAAV, scAAV was selected because of the overall improved transgene expression of its vectors compared to ssAAV vectors (Bogner et al., 2015; Borras et al., 2006). eGFP was selected as a transgene because of the ease of quantitating transgene product fluorescence by flow-cytometry and because live cultures can be analyzed by microscopy. All scAAV vectors used in these studies are manufactured using protocols established at the University of Iowa with good manufacturing practices (GMP). Human primary TM cell strains (n=2 each from normal and glaucoma donors) and murine primary TM cell strains (n=2 each from wild type and Tg-MYOCY437H mice) were transduced. The reason for selecting human glaucomatous TM cell strains and mouse TM cells from a glaucoma model is to test AAV(s) transduction efficiency in diseased cells with possible different physiology, different cell surface receptors and which are the final target cells for human use. To avoid heterologous mixtures of cells, the primary cell types are isolated and characterized, as described previously (Mao et al., 2013; Stainer et al., 2000; Keller et al., 2018). For initial screening, all cell lines are infected at two MOI of 2500 and 10,000 vector genomes/cell and eGFP expression will be examined via fluorescent microscopy and flow-cytometry 72 hours post infection as shown in FIG. 1. Higher MOI ensures that if a cell was not transduced it was not because of an ineffective low MOI.

Determine the most selective AAV vector(s) for transducing mouse TM cells in vivo. Briefly, mice are injected with each of the 9 AAV serotypes intravitreally (2 μl of 1013 VG/ml). The contralateral eye is injected with the same virus (at the same titer) but intracamerally. Commencing on day 4 after the injection and then on following predetermined days, each injected eye will be examined under ultraviolet illumination for fluorescence, as an indication of the presence of GFP in the anterior chamber (Miller et al., 2008). Based on its intensity and distribution, fluorescence grades from 1 to 5 (with 5 being the strongest intensity) are assigned at the indicated days by a researcher who will be masked to the treatments (Miller et al., 2008). Based on preliminary data and power calculations, 6 mice per serotype allow for 0.90 power to observe a significant difference (p<0.05) of 2 grades between viral serotypes and injection mode with a S.D. of 1 grade. Three mice of each sex are employed. Power calculations indicate that this gives about 0.70 power to detect differences between sexes. Throughout the study, all eyes (injected and control) are carefully examined with a hand-held ophthalmoscope and slit lamp for the presence of iridial hyperemia and lenticular opacity. Animals are also assessed daily for potential gross abnormalities in appearance and behavior by the veterinary staff. At the end a two-week post-treatment period, enucleated eyes are fixed and cryo-sectioned. The sections are evaluated by fluorescence microscopy for evidence of GFP expression in various ocular tissues (corneal endothelium, iris, TM, lens, ciliary body, sclera, retina and optic nerve head) as shown in FIG. 1. H and E staining and transmission electron microscopy (TEM) are used to evaluate the effects of different AAVs on the TM integrity and ultrastructure between two different routes of injections. At least the AAV serotype with the most selective tropism for TM (even if it does not have the highest transduction efficiency) is employed for further work.

Determine the most selective AAV vector(s) for transducing human TM cells cx vivo. Fresh human donor eyes are obtained from regional eye banks. AAV serotypes identified above are injected into the perfusate as described previously (Clark et al., 1995). The other eye of the pair is injected with control AAV (no GFP). Transduction efficiency is examined by qPCR, Western blot analysis and immunohistochemistry for evidence of GFP expression in various ocular tissues (TM, corneal endothelium, sclera). H and E staining and TEM are used to evaluate the effects of AAVs on the TM integrity and ultrastructure compared to control injections. The same clinical scoring system is used to detect fluorescence intensity n=5 for each vector examined should allow for obtaining statistical significance.

Generate and test selected AAVs expressing CRISPR-Cas9 targeting MYOC in primary human TM cells. AAVs expressing Cas9 and guide RNAs (gRNAs) targeting MYOC were prepared. Primary TM cells are transduced with AAV expressing Cas9 alone (control) or Cas9 and gRNA targeting MYOC at MOI of 2500 and 10,000 VG/cell. As a negative control, TM cells are transduced with AAV expressing Cas9 and non-targeting gRNA sequences. SURVEYOR assays and/or Sanger sequencing are performed to detect the percentage of indels created as described previously (Jin et al., 2017). Western blotting and immunostaining will be used to confirm knockdown efficiency at the protein level. Conditioned medium and cell lysates are analyzed for ER stress and ECM markers using Western blotting.

Determine off-target mutation frequencies in human perfusion organ cultured (POC) anterior segments using GUIDE-Sea. The CRISPR-Cas9 system can tolerate mismatches in the guide target sequences such that off-target mutagenesis in treated cells presents a potential safety concern. To delineate genome-wide off-target cleavage in the above described constructs targeting MYOC in an unbiased manner, the GUIDE-Seq method (Tsai et al., 2014) is applied. Briefly, whole human donor eyes will be dissected to remove retina, ciliary body and iris. Anterior segment containing the TM and cornea are set-up for perfusion culture and are treated with AAV-CR-MYOC individually. After 2-weeks of perfusion culture, TM are isolated and subjected to sequencing. From each eye, retina, ciliary body and iris tissues (obtained prior to being exposed to genome editing) are used as a control. CRISPR-Cas9-induced double-stranded breaks (DSBs) are tagged by integrating blunt, double-stranded oligodeoxynucleotides. Subsequently, the tags are precisely mapped using next-generation sequencing and bioinformatic analysis. Bioinformatic analysis is performed by the University of Iowa NEI-funded P30 Analytical Core Facility.

### Results

Clinical grade AAVs with high titers are prepared. Different AAV serotypes likely have different transduction efficiencies and specificities for the target tissue (TM). It is likely that the intracameral route results in efficient transduction of TM by AAV (based on the anatomy), but intravitreal injections result in a longer-lasting effect (depot effect for sustained release). As mentioned above, the AAV serotype with the most selective tropism for TM (even if it does not have the highest transduction efficiency) is used in subsequent studies. Engineered serotypes (e.g., AAV1.3 which is a hybrid of AAV1 and AAV6) may be employed. This gives us the opportunity to screen among numerous possible choices. Non-viral delivery methods may be employed (Timin et al., 2018; Wang et al., 2018). A single injection of AAV may suffice to transduce the anterior segment POC system. Recent studies have shown that CRISPR-Cas9 off-target mutations are rare (Iyer et al., 2018, Iyer et al., 2015; CRISPR, 2018). However, Cas9 can be re-designed to reduce off-target effects (Doench et al., 2016; Merienne et al., 2017).

### Data

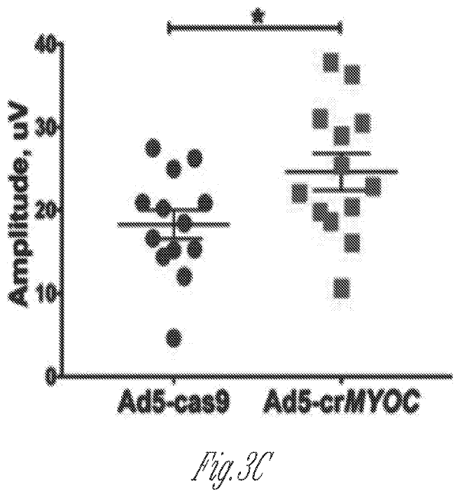

Ad5.cr MYOC mediated IOP lowering in mouse eyes: As shown in FIG. 3, the human mutant myocilin transgene was targeted with Ad5-crMYOC virus injected intravitreally (2×10⁷ pfu/eye). Ad5-crMYOC treatment prevented IOP elevation compared with Ad5-cas9-treated eyes (Jain et al., 2017). Targeting myocilin with Ad5-crMYOC also lowered high IOP in a cohort of older Tg-MYOC^{Y437H} mice (age 29 months) as early as 2 weeks post-injection (FIG. 3A). There was a significant increase in outflow facility 1 month after Ad5-crMYOC treatment in a separate cohort of 4 to 5-mo-old Tg-MYOC^{Y437H} mice (FIG. 3B). The improved TM health and rescue in the IOP phenotype in Ad5-crMYOC-treated eyes correlated with the significantly improved RGC function in Tg-MYOC^{Y437H} mice (FIG. 3C).

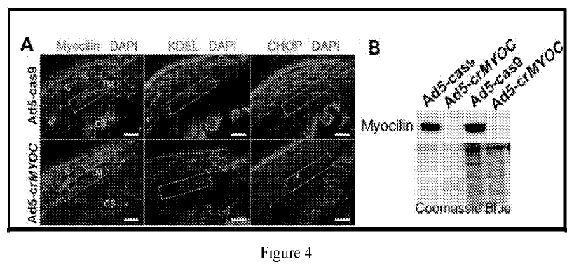

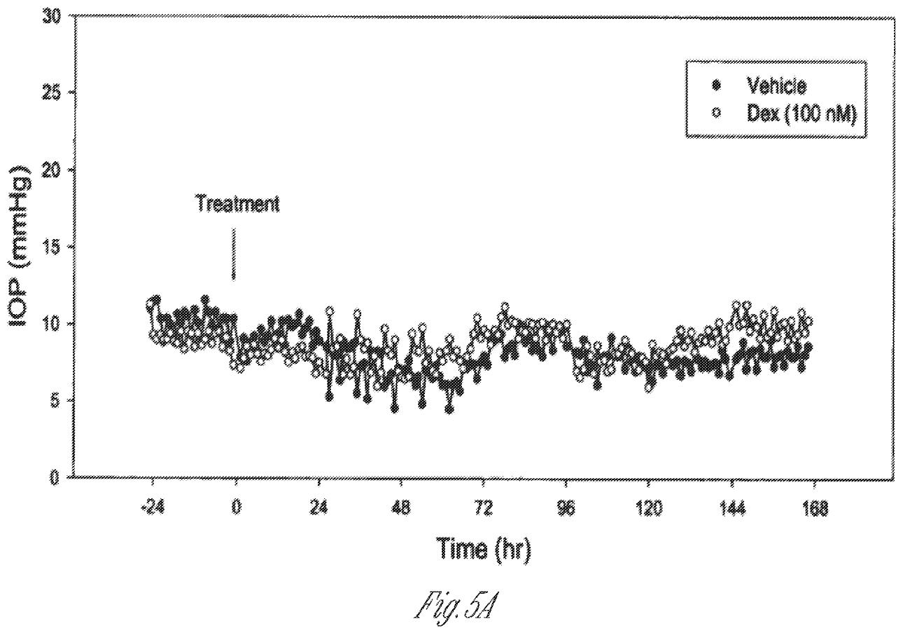

Efficient and selective myocilin knockdown via CRISPR-Cas9 in vivo in mice and ex vivo in human anterior segments: CRISPR gRNA (for MYOC) is both efficient and selective in vitro and in vivo (Jain et al., 2017). Fixed mouse eyes treated with Ad5-crMYOC were immunostained with myocilin and ER stress markers (FIG. 4A). TM tissue sections from Ad5-crMYOC-treated eyes showed reduced myocilin, KDEL (GRP78 and GRP94), and CHOP labeling, indicating that the gRNA is targeting MYOC in vivo (Top panel) compared to AD5-Cas9 treated mice (FIG. 4A). The feasibility of using genome editing to treat glaucoma in human TM tissues was investigated using a human ex vivo POC anterior segment. Ad5-crMYOC reduced myocilin mRNA (Jain et al., 2017) and myocilin secretion into the perfusate medium obtained from the TM outflow pathway compared with the contralateral fellow eye treated with Ad5-cas9 (FIG. 4B), further supporting the ex vivo efficiency of Ad5-crMYOC in perfusion-cultured human eyes.

### Experimental Design

Knockdown of mutant human MYOC in Tg-MYOC^{Y437H} mice: Intraocular injections of AAV expressing Cas9 and a guide RNA targeting the 5′ coding region of the mutant human MYOC in adult Tg-MYOC^{Y437H} mice were performed after first determining the most efficient and specific AAV for targeting the TM. The site of injection may be intracameral or intravitreal. Tg-MYOC^{Y437H} mice develop elevated IOP starting at 3-months of age (Zode et al., 2011). Elevation of IOP in young Tg-MYOC^{Y437H} mice as well as lower IOP in aged Tg-MYOC^{Y437H} mice can be prevented by targeting MYOC using CRISPR (Jain et al., 2017) (FIGS. 3 and 4). AAV expressing Cas9 and gRNA are used to target mutant MYOC in the anterior chamber. The contralateral eye is injected with control AAV (expressing just Cas9). A cohort of older mice (4-6 months old) are injected with either AAV expressing Cas9 and gRNA targeting MYOC or control AAV virus. The knockdown efficiency of mutant MYOC is determined by qPCR for mRNA and Western blotting for protein levels in the iridocorneal angle tissues of Tg-MYOC^{Y437H} mice. Deep sequencing is employed to assess the efficiency of DNA editing at the mutant MYOC locus in the TM.

Examination of glaucoma phenotypes in Tg-MYOC^{1437H} mice: IOPs in Tg-MYOC^{Y437H} mice are evaluated after 1, 2, and 3 months of treatment. It is determined whether AAV expressing Cas9 and gRNA targeting MYOC treatment prevents IOP elevation and lowers IOP, 5-month old Tg-MYOC^{Y437H} mice show reduced retinal ganglion cell (RGC) function using pattern electroretinography (PERG) compared to WT littermates (Zode et al., 2011). It is determined whether Tg-MYOC^{Y437H} mice treated with AAV-Cas9 targeting MYOC show an improved PERG post treatment. At the end of the experiment, levels of the unfolded protein response (UPR) and ER stress markers in TM are analyzed by RT-PCR, Western immunoblotting, and immunohistochemistry. Based on preliminary data, prior studies and power calculations based on previous differences observed in IOP, 8 mice per group to have 0.98 power to observe a significant difference (p<0.05) of 4.0 mmHg (S.D.=2.0 mmHg) between treated and untreated mice. 8 mice (4 of each sex) per group are used in order to ensure ample power to detect significant differences. Use of 4 animals per sex per group will provide 0.80 power to detect significant differences between the sexes.

Utilize the human anterior segment organ culture system to test the ability to efficiently perform genome editing on TM tissue: Fresh human donor eyes are obtained from regional eye banks. AAV containing the Cas9 nuclease system targeting MYOC is injected in the perfusate as described previously (Clark et al., 1995). Efficiency of knockdown is examined by qPCR and Western blot analysis. H and E staining and TEM is used to evaluate the effects of Cas9 on the TM integrity compared to control injections (AAV expressing just Cas9). n=5 pairs of eyes is sufficient for statistically significant data. If necessary, additional eyes will be added to obtain significance based on power calculations.

### Results

Intraocular injections of AAV expressing Cas9 targeted to mutant human MYOC result in efficient editing of the mutant MYOC transgene. The edited locus leads to knockdown of mutant myocilin and associated ER stress in adult Tg-MYOC^{Y437H} mice and reduction of IOP in adult Tg-MYOC^{Y437H} mice. Early targeting of mutant MYOC with the AAV-CRISPR system likely prevents elevation of IOP in young Tg-MYOC^{Y437H} mice. This reduced IOP was beneficial effects on retinal ganglion cells health and resultant RGC function measured by pattern ERG.

### Data

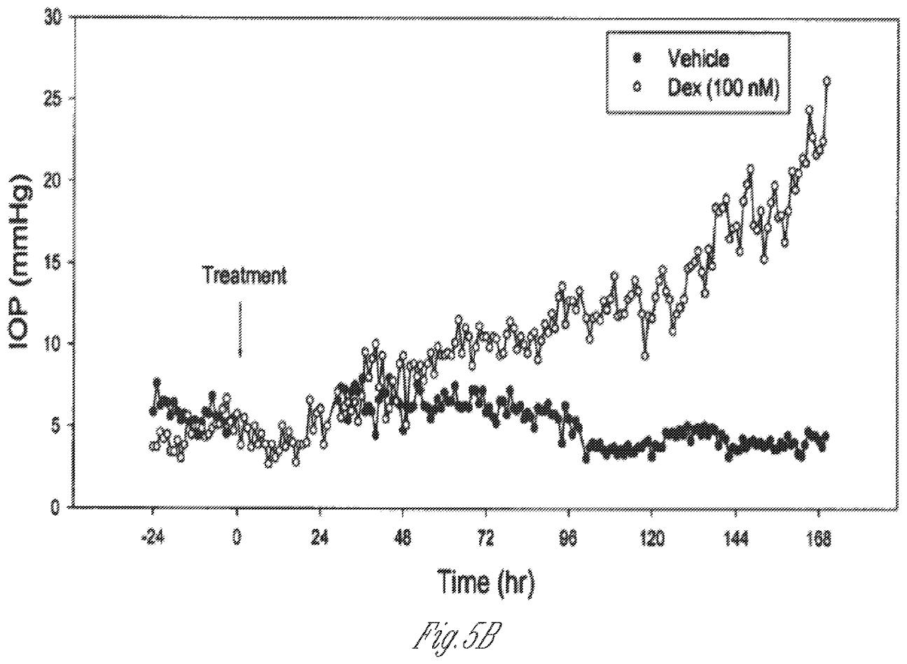

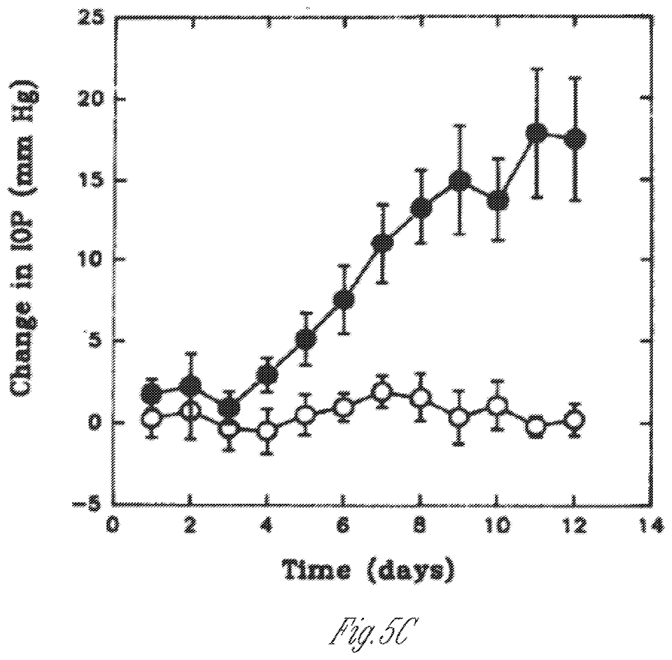

DEX-induced ocular hypertension in ex-vivo perfusion organ cultured human donor eyes: A DEX-induced ocular hypertension ex-vivo model was developed (FIG. 5). Approximately 40% of the eyes perfusion cultured with DEX (100 nM) develop significant increases in IOP due to an impaired aqueous humor outflow facility (Clark et al., 1995).

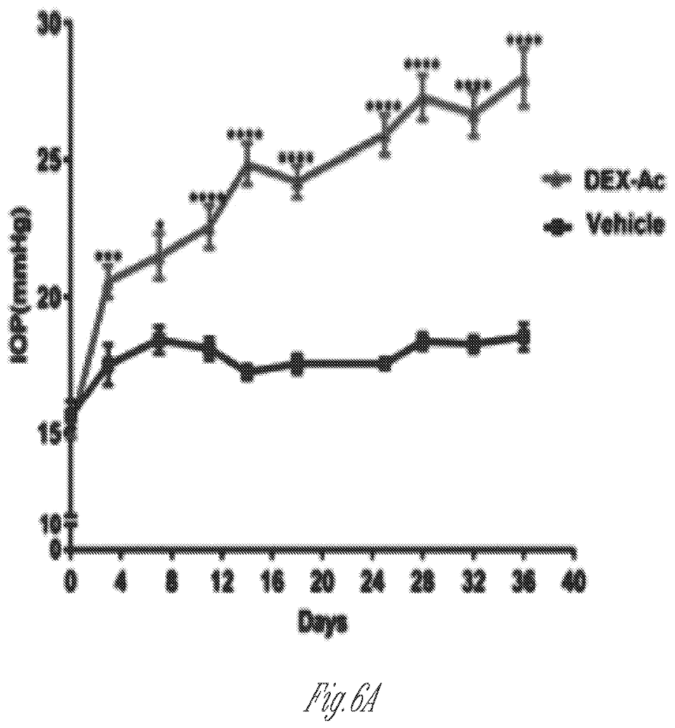

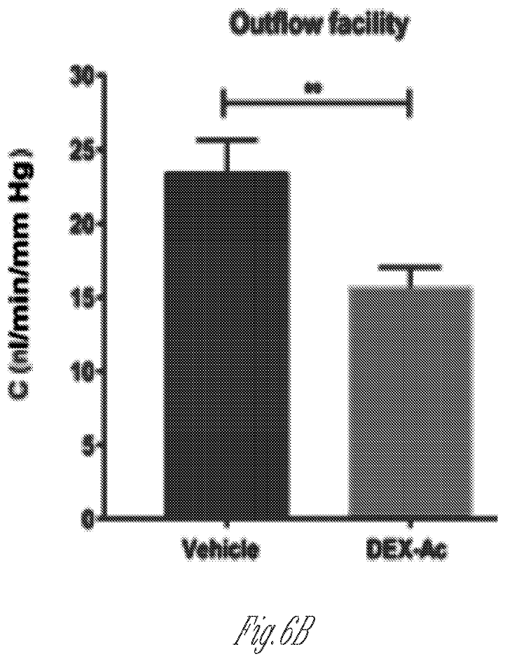

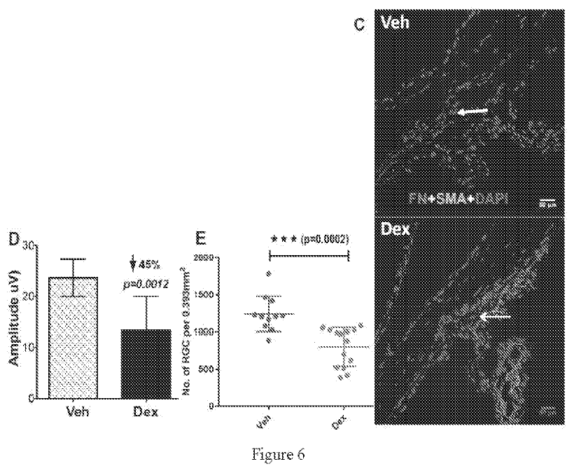

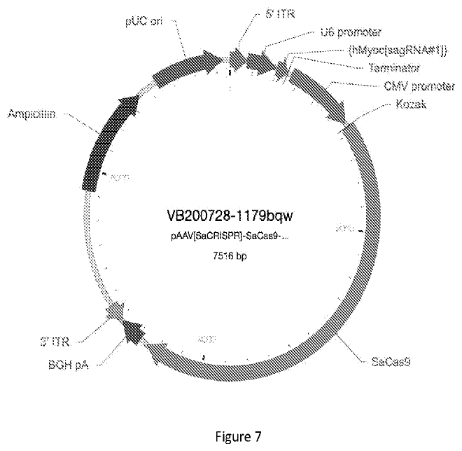

Development of a mouse model of GC-induced glaucoma. A model of GC-induced glaucoma was prepared by weekly periocular (PO) injections of DEX acetate suspension (Patel et al., 2017). DEX treatment led to sustained and significantly elevated IOP (FIG. 6A). Similar to POAG, DEX-induced IOP elevation is associated with reduced outflow facility (FIG. 6B). Conventional outflow facility was significantly reduced (33%) in DEX-treated mice compared to Veh-treated mice (p=0.004; n=12). Immunostaining for fibronectin and a-smooth muscle actin (SMA) revealed that both fibronectin and SMA were remarkably increased in the TM of 5-week DEX-treated mice compared to Veh-treated mice (FIG. 6C). In addition, we reported that DEX increases collagen I in the TM (Patel et al., 2017). Since PO injection of DEX led to sustained and prominent IOP elevation, the functional and structural loss of RGCs were examined in 10-12 weeks DEX-treated mice by PERG (FIG. 6D) and whole mount retina staining with RBPMS (FIG. 6E) as described in Zode et al. (2011); Jain et al. (2017). In preliminary studies, there was an about 45% loss of PERG amplitudes indicating functional deficit in RGCs of 10-weeks DEX-treated mice. DEX-treated mice also demonstrated about 36% loss of RGCs compared to Veh-treated mice.

### Example II

Primary open angle glaucoma (POAG) is a common cause of irreversible blindness worldwide. In 1997 Stone, Sheffield and colleagues identified mutations in the myocilin (MYOC) gene (previously referred to as trabecular meshwork-induced glucocorticoid response gene or TIGR) in families with autosomal dominant POAG (Stone et al., 1997). Subsequent work has revealed that mutations in MYOC account for approximately 3% of POAG cases (Alward et al., 1998, Fingert et al., 1999). MYOC has three exons and encodes a 504 amino acid polypeptide that contains two major homology domains, an N-terminal myosin-like domain and a C-terminal olfactomedin-like domain (Fingert et al., 1998). Numerous glaucoma-causing mutations, as well as neutral polymorphisms have been identified. The vast majority of disease-causing mutations are found in the olfactomedin-like domain in the third exon. Most disease-causing mutations are missense mutations, with one particularly common truncating mutation GLN368Ter (Q368X) (Stone et al., 1997; Alward et al., 1998; Fingert et al., 1999). Evidence indicates that haploinsufficiency of myocilin does not cause POAG, but rather mutation of one copy of the myocilin protein results in protein misfolding leading to protein aggregation and endoplasmic reticulum stress (Jacobson et al., 2001; Zode et al., 2001). This gain-of-function mechanism leads to abnormal trabecular meshwork function causing elevated intraocular pressure, and eventually retinal ganglion cell death and visual field loss (Jacobson et al., 2001; Zode et al., 2001; Zain et al., 2017). Of interest, it has also been determined that myocilin has a mutation-dependent gain-of-function related to a peroxisomal targeting signal (PTS1) composed of the COOH-terminal three amino acids (SKM). The PTS1 signal appear to be exposed when one copy of the myocilin protein contains a missense mutation, or one copy of a truncated protein (such as GLN368Ter) forms a dimer with wild-type myocilin (containing the C-terminal SKM) (Shpeard et al., 2007). Absence of the C-terminal SKM sequence can be protective of glaucoma mutations. It has been shown that mutation of the serine 502 residue to proline eliminates the PTS1 signal (SER502PRO) (Shepard et al., 2007).

The present system may be employed for gene editing of MYOC to treat glaucoma by targeting the trabecular meshwork with CRISPR-Cas9 guide RNAs that perform one of the following: 1) creation of a premature termination upstream (towards the N-terminus) of the specific MYOC mutation being treated. Since nearly all MYOC mutations are in exon 3, we claim the creation of a premature termination codon or a frameshift (leading to a premature termination codon) upstream of the known GLN368Ter mutation in exon 3 (i.e., in exon 1 or exon 2) will treat POAG, or 2) creation of a C-terminal mutation leading to the alteration of the SKM PTS1 signal sequence, such as the SER502PRO mutation.

### Example III

Any Cas protein and matched gRNA may be employed in the vectors and methods described herein. For example. Cas-phi has PAM=TTA. guide RNAs for that Cas useful to target myocilin include but are not limited to TGGCCTGCCTGGTGTGGGAT (SEQ ID NO: 10), CAGAGAGACAGCAGCACCCA (SEQ ID NO:11), GACCTGGAGGCCACCAAAGC (SEQ ID NO:12), TGGAGGAAGAGAAGAAGCGA (SEQ ID 140:13), CAGGCCATGTCAGTCATCCA (SEQ ID NO:14) or GGAGGAAGAGAAGAAGCGAC (SEQ ID NO:15 or a nucleotide sequence with at least 90%, 92%, 95%, 96%, 98% or more nucleotide sequence identity thereto.

### REFERENCES

Acland et al., Mol. Ther., 12:1072 (2005).
AGIS, Am. J. Ophthalmol., 30:429 (2000).
Alward et al., N. Engl. J. Med., 338:1022 (1998).
Bainbridge et al., N. Engl. J. Med., 372:1887 (2015).
Bogner et al., PloS one 10:e0128759 (2015).
Bollinger et al., Mol. Vis., 18:2001 (2012).
Borras et al., J. Gene Med., 8:589 (2006).
Bove et al., Mol. Ther., 21:509 (2013).
Chen et al., Viruses, 10:______ (2018).
Clark & Wordinger, Exp. Eye. Res., 88:752 (2009).
Clark et al., Cell Motil. Cytoskeleton, 60:83 (2005).
Clark et al., Invest. Ophthalmol. Vis. Sci., 35:281 (1994).
Clark et al., Invest. Ophthalmol. Vis. Sci., 36:478 (1995).
Clark, J. Glaucoma. 4:354 (1995).
Cong et al., Science, 339:819 (2013).
CRISPR, Nature Methods, 15:229 (2018).
Doench et al., Nature Biotechnology, 34:184 (2016).
Ellis et al., Virol. J., 10:74 (2013).
Fingert et al., Hum. Mol. Genet., 8:899 (1999).
Fingert et al., Genome Res., 377 (1998).
Fleenor et al., Invest. Ophthalmol. Vis. Sci., 47:226 (2006).
Fuchshofer et al., Exp. Eye Res., 77:757 (2003).
Heijl et al., Arch. Ophthalmol., 120:1268 (2002).
Hetz et al., Nature Cell Biol., 17:829 (2015).
Hickey et al., Gene Ter., 24:787 (2017).
Hsu et al., Nature Biotechnology. 31:827 (2013).
Inatani et al., Graefes Arch. Clin. Exp. Ophthalmol., 239:109 (2001).
Iyer et al., Nat. Methods, 12:479 (2015).
Iyer et al., PLoS Genet., 14:e1007503 (2018).
Jacobson et al., Arch. Ophthalmol., 130:9 (2012).
Jacobson et al., Hum. Mol. Genet. 10:117 (2001).
Jain et al., Proc. Natl. Acad. Sci. USA, 114:11199 (2017).
Joe et al., Biochem. Biophys. Res. Commun., 312:592 (2003).
Joseph et al., Semin. Ophthalmol., 32:86 (2017).
Kasetti et al., Invest. Ophthalmol. Vis. Sci., 57:6058 (2016).
Kasetti et al., J. Biol. Chem., 293:9854 (2018).
Kass et al., Arch. Ophthalmol., 120:701 (2002).
Kaur et al., Curr. Cardiol. Rep., 20:58 (2018).
Keller et al., Exp. Eye Res., 171164 (2018).
Lutjen-Drecoll, Exp. Eye Res., 81:1 (2005).
Mali et al., Science, 339:823 (2013).
Mao et al., Invest. Ophthalmol Vis. Sci., 54:3600 (2013).
Mao et al., Invest. Ophthalmol. Vis. Sci., 52:8068 (2011).
McDowell et al., Exp. Eye Res., 116:419 (2013).
Merienne et al., Cell Rep., 20:2980 (2017).
Millar et al., Mol. Vis., 4:10 (2008).
Mollanoori & Teimourian, Biotechnol. Lett., 40:907 (2018).
Montecchi-Palmer et al., Invest. Ophthalmol. Vis. Sci., 58:1288 (2017).
Ochiai & Ochiai, Jpn. J. Ophthalmol., 46:249 (2002).
Oyadomari et al., Cell Death Differ., 11:381 (2004).
Pang et al., Exp. Eye Res., 73:815 (2001).
Pang et al., J. Glaucoma. 9:468 (2000). PubMed PMID: 11131754.
Patel et al., Am. J. Pathol., 187:713 (2017).
Peters et al., Am. J. Ophthalmol., 156:724 (2013).
Peters et al., Invest. Ophthalmol. Vis. Sci. 56:3860 (2015).
Picht et al., Graefes Arch. Clin. Exp. Ophthalmol., 239:199 (2001).
Raghunathan et al., Invest. Ophthalmol. Vis. Sci., 56:4447 (2015).
Ran et al., Nat. Protoc., 8:2281 (2013).
Razeghinejad & Katz. Ophthalmic Res., 47:66 (2012).
Razzouk, Ann. Hum. Genet., ______:______ (2018).
Shepard et al., Invest. Ophthalmol. Vis. Sci., 51:2067 (2010).
Shepard et al., Hum Mol Genet., 16:609 (2007)
Stainer et al., Curr. Eye Res., 20:347 (2000).
Stone et al., Science, 225:668 (1997).
Tham et al., Ophthalmology. 121:2081 (2014).
Timin et al., Nanomedicine. 14:97 (2018).
Tovar-Vidales et al., Exp. Eye Res., 93:442 (2011).
Tsai et al., Nature Biotechn., 33:187 (2014).
Wang et al., PloS one. 12:e0182473 (2017).
Wang et al., Proc. Natl. Acad. Sci. USA, 115:4903 (2018).
Zode et al., Clin. Invest., 121:3542 (2011).
Zode et al., J. Clin. Invest., 124:1956 (2014).

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

1. An isolated nucleic acid vector comprising a first expression cassette comprising a first promoter operably linked to an open reading frame for a Cas recombinase and a second expression cassette comprising a second promoter operably linked to a nucleotide sequence comprising a myocilin specific gRNA, wherein the vector is not an adenoviral vector or an isolated nucleic acid vector comprising a first expression cassette comprising a first promoter operably linked to an open reading frame for a Cas recombinase and a second expression cassette comprising a second promoter operably linked to a nucleotide sequence comprising a gRNA having SEQ ID NO:1 or SEQ ID NO:2.

2. (canceled)

3. The vector of claim 1 which is a viral vector.

4. The vector of claim 3 which is an adeno-associated virus (AAV), retrovirus or lentivirus vector.

5. The vector of claim 1 wherein the Cas is saCas9.

6. The vector of claim 1 wherein the gRNA comprises SEQ ID NO:1.

7. The vector of claim 1 wherein the gRNA comprises SEQ ID NO:2.

8. The vector of claim 1 wherein the first promoter is a heterologous RNA polymerase I promoter.

9. The vector of claim 1 wherein the second promoter is a RNA polymerase III promoter.

10. The vector of claim 1 which is in a nanoparticle.

11. The vector of claim 10 wherein the nanoparticle comprises a liposome.

12. A method to prevent, inhibit or treat glaucoma, to decrease intraocular pressure or to reduce MYOC expression in a mammal, comprising: administering to the mammal an effective amount of a composition having the vector of claim 1.

13-14. (canceled)

15. The method of claim 12 wherein the mammal is a human.

16. The method of claim 12 wherein the composition is injected into the anterior segment of the eye, to trabecular meshwork cells, intracamerally or intravitreally.

17-19. (canceled)

20. The method of claim 12 wherein the administration results inactivation of at least one myocilin gene or expression of a truncated myocilin.

21. (canceled)

22. The method of claim 12 wherein the vector is a viral vector.

23. The method of claim 22 wherein the viral vector is an adeno-associated viral vector.

24. The method of claim 23 wherein the capsid is an AAV2, AAV5 or AAV9 capsid or wherein the genome is a rAAV2 genome.

25. (canceled)

26. The method of claim 12 wherein the composition is a liposome comprising the vector.

27. The method of claim 12 wherein the mammal has primary open angle glaucoma.


SEQUENCE LISTING (15 sequences)

SEQ ID NO: 1 - DNA, 21 bp, Artificial Sequence
  A synthetic sequence
gtcagtcatccataacttaca

SEQ ID NO: 2 - DNA, 22 bp, Artificial Sequence
  A synthetic sequence
gaccagctggaaacccaaacca

SEQ ID NO: 3 - DNA, 21 bp, Artificial Sequence
  A synthetic sequence
ggctccagagaagtttctacg

SEQ ID NO: 4 - skipped

SEQ ID NO: 5 - DNA, 20 bp, Artificial Sequence
  A synthetic sequence
ggcctgcctggtgtgggatg

SEQ ID NO: 6 - DNA, 1934 bp, Homo sapiens
cgggccccggacacccgctctgcacagcagagctttccagaggaagcctcaccaagcctctgcaatgaggttcttctgtgcacgttgctgcagctttgggcctgagatgccagctgtccagctgctgcttctggcctgcctggtgtgggatgtgggggccaggacagctcagctcaggaaggccaatgaccagagtggccgatgccagtataccttcagtgtggccagtcccaatgaatccagctgcccagagcagagccaggccatgtcagtcatccataacttacagagagacagcagcacccaacgcttagacctggaggccaccaaagctcgactcagctccctggagagcctcctccaccaattgaccttggaccaggctgccaggccccaggagacccaggaggggctgcagagggagctgggcaccctgaggcgggagcgggaccagctggaaacccaaaccagagagttggagactgcctacagcaacctcctccgagacaagtcagttctggaggaagagaagaagcgactaaggcaagaaaatgagaatctggccaggaggttggaaagcagcagccaggaggtagcaaggctgagaaggggccagtgtccccagacccgagacactgctcgggctgtgccaccaggctccagagaagtttctacgtggaatttggacactttggccttccaggaactgaagtccgagctaactgaagttcctgcttcccgaattttgaaggagagcccatctggctatctcaggagtggagagggagacaccggatgtggagaactagtttgggtaggagagcctctcacgctgagaacagcagaaacaattactggcaagtatggtgtgtggatgcgagaccccaagcccacctacccctacacccaggagaccacgtggagaatcgacacagttggcacggatgtccgccaggtttttgagtatgacctcatcagccagtttatgcagggctacccttctaaggttcacatactgcctaggccactggaaagcacgggtgctgtggtgtactcggggagcctctatttccagggcgctgagtccagaactgtcataagatatgagctgaataccgagacagtgaaggctgagaaggaaatccctggagctggctaccacggacagttcccgtattcttggggtggctacacggacattgacttggctgtggatgaagcaggcctctgggtcatttacagcaccgatgaggccaaaggtgccattgtcctctccaaactgaacccagagaatctggaactcgaacaaacctgggagacaaacatccgtaagcagtcagtcgccaatgccttcatcatctgtggcaccttgtacaccgtcagcagctacacctcagcagatgctaccgtcaactttgcttatgacacaggcacaggtatcagcaagaccctgaccatcccattcaagaaccgctataagtacagcagcatgattgactacaaccccctggagaagaagctctttgcctgggacaacttgaacatggtcacttatgacatcaagctctccaagatgtgaaaagcctccaagctgtacaggcaatggcagaaggagatgctcagggctcctggggggagcaggctgaagggagagccagccagccagggcccaggcagctttgactgctttccaagttttcattaatccagaaggatgaacatggtcaccatctaactattcaggaattgtagtctgagggcgtagacaatttcatataataaatatcctttatcttctgtcagcatttatgggatgtttaatgacatagttcaagttttcttgtgatttggggcaaaagctgtaaggcataatagtttcttcctgaaaaccattgctcttgcatgttacatgggtaccacaagccacaataaaa

SEQ ID NO: 7 - protein, 504 aa, Homo sapiens
MRFFCARCCSFGPEMPAVQLLLLACLVWDVGARTAQLRKANDQSGRCQYTFSVASPNESSCPEQSQAMSVIHNLQRDSSTQRLDLEATKARLSSLESLLHQLTLDQAARPQETQEGLQRELGTLRRERDQLETQTRELETAYSNLLRDKSVLEEEKKRLRQENENLARRLESSSQEVARLRRGQCPQTRDTARAVPPGSREVSTWNLDTLAFQELKSELTEVPASRILKESPSGYLRSGEGDTGCGELVWVGEPLTLRTAETITGKYGVWMRDPKPTYPYTQETTWRIDTVGTDVRQVFEYDLISQFMQGYPSKVHILPRPLESTGAVVYSGSLYFQGAESRTVIRYELNTETVKAEKEIPGAGYHGQFPYSWGGYTDIDLAVDEAGLWVIYSTDEAKGAIVLSKLNPENLELEQTWETNIRKQSVANAFIICGTLYTVSSYTSADATVNFAYDTGTGISKTLTIPFKNRYKYSSMIDYNPLEKKLFAWDNLNMVTYDIKLSKM

SEQ ID NO: 8 - DNA, 1228 bp, Homo sapiens
gatctccagttcctagcatagtgcctggcacagtgcaggttctcaatgagtttgcagagtgaatggaaatataaactagaaatatatccttgttgaaatcagcacaccagtagtcctggtgtaagtgtgtgtacgtgtgtgtgtgtgtgtgtgtgtgtgtaaaaccaggtggagatataggaactattattggggtatgggtgcataaattgggatgttctttttaaaaagaaactccaaacagacttccggaaggttattttctaagaatcttgctggcagcgtgaaggcaacccccctgtgcacagccccacccagcctcacgtggccacctctgtcttcccccatgaagggctggctccccagtatatataaacctctctggagctcgggcatgagccagcaaggccacccatccaggcacctctcagcacagcagagctttccagaggaagcctcaccaagcctctgcaatgaggttcttctgtgcacgttgctgcagctttgggcctgagatgccagctgtccagctgctgcttctggcctgcctggtgtgggatgtgggggccaggacagctcagctcaggaaggccaatgaccagagtggccgatgccagtataccttcagtgtggccagtcccaatgaatccagctgcccagagcagagccaggccatgtcagtcatccataacttacagagagacagcagcacccaacgcttagacctggaggccaccaaagctcgactcagctccctggagagcctcctccaccaattgaccttggaccaggctgccaggccccaggagacccaggaggggctgcagagggagctgggcaccctgaggcgggagcgggaccagctggaaacccaaaccagagagttggagactgcctacagcaacctcctccgagacaagtcagttctggaggaagagaagaagcgactaaggcaagaaaatgagaatctggccaggaggttggaaagcagcagccaggaggtagcaaggctgagaaggggccagtgtccccagacccgagacactgctcgggctgtgccaccaggctccagagaaggtaagaatgcagagtggggggactctgagttcagcaggtgatatggctcgtagtgacctgctacaggcgctccaggcctccctgcctgccctttctcctagagactgcacagctagcacaagacagatgaattaaggaaagcacagcgatc

SEQ ID NO: 9 - skipped

SEQ ID NO: 10 - DNA, 20 bp, Artificial Sequence
  A synthetic sequence
tggcctgcctggtgtgggat

SEQ ID NO: 11 - DNA, 20 bp, Artificial Sequence
  A synthetic sequence
cagagagacagcagcaccca

SEQ ID NO: 12 - DNA, 20 bp, Artificial Sequence
  A synthetic sequence
gacctggaggccaccaaagc

SEQ ID NO: 13 - DNA, 20 bp, Artificial Sequence
  A synthetic sequence
tggaggaagagaagaagcga

SEQ ID NO: 14 - DNA, 20 bp, Artificial Sequence
  A synthetic sequence
caggccatgtcagtcatcca

SEQ ID NO: 15 - DNA, 20 bp, Artificial Sequence
  A synthetic sequence
ggaggaagagaagaagcgac